(19) United States
(12) Patent Application Publication
(10) Pub. No.: US 2025/0210153 A1
(43) Pub. Date: Jun. 26, 2025

(54) METHOD FOR DETERMINING A BIODEGRADABILITY OF A POLYMER

(71) Applicant: BASF SE, Ludwigshafen am Rhein (DE)

(72) Inventors: Volker Settels, Ludwigshafen (DE); Jessica Eleanor Bean, Ludwigshafen (DE); Andreas Buechse, Ludwigshafen (DE); Ning Wang, Ludwigshafen (DE); Sandra Gonzalez Maldonado, Ludwigshafen (DE); Glauco Battagliarin, Ludwigshafen (DE); Michael Bernhard Schick, Ludwigshafen (DE); Andreas Kuenkel, Ludwigshafen (DE)

(21) Appl. No.: 18/838,361

(22) PCT Filed: Feb. 17, 2023
(86) PCT No.: PCT/EP2023/054074

§ 371 Date: Aug. 14, 2024

(30) Foreign Application Priority Data
Feb. 18, 2022 (EP) 22157567.3
Feb. 18, 2022 (EP) 22157573.1
Feb. 18, 2022 (EP) 22157580.6

(51) Int. Cl. G16C 60/00; G06F 30/27; G06F 113/26; G06F 119/04; G16C 20/10; G16C 20/30; G16C 20/70

(52) U.S. Cl. CPC G16C 60/00; G06F 30/27; G16C 20/10; G16C 20/30; G16C 20/70; G06F 2113/26; G06F 2119/04

(57) ABSTRACT

The invention refers to a method for determining a biodegradability for a polymer. A digital representation of the polymer indicative of physicochemical characteristics of the polymer is provided. Further, a habitat is provided that is indicative of habitat descriptor values influencing a biodegradation of a polymer. The habitat descriptors are indicative of environmental characteristics of the habitat. A biodegradation model is provided based on the habitat. wherein the biodegradation model is adapted to determine a biodegradability of a polymer in the respective habitat, wherein the biodegradation model is a data driven model parametrized with respect to the habitat such that it can determine a biodegradability of a polymer based on the physicochemical characteristics. The biodegradability of the polymer is then determined based on the provided biodegradation model and the digital representation of the polymer.

### FIELD OF THE INVENTION

The invention relates to a method, an apparatus and a computer program product for determining a biodegradability usable for validating a biodegradation of a polymer. Further, the invention refers to a training method, a training apparatus and a training computer program for training a data driven biodegradation model utilizable by the method, apparatus and computer program product for determining the biodegradability of a polymer. Moreover, the invention refers to a method and apparatus for providing an interface for the determination of the biodegradability of a polymer.

### BACKGROUND OF THE INVENTION

Generally, polymers are widely used in industrial and/or daily use products due to their broad range of application properties. The use of polymers encompasses amongst others coatings, personal care products, washing detergents, lubricants, packages and foams. However, this widely spread application leads to a huge amount of waste containing the used polymers. While it is in fact in most cases the durability of the polymers that makes them popular for many uses, exactly this durability leads to a plurality of problems in waste management, in particular, since the polymers are also durable in waste. In particular, if non-biodegradable polymers are not suitably collected in intended waste stream, this can result in increased micro-plastic contamination and bioaccumulation in the environment. Thus, there is not only a need for polymers that decompose, but also a need to take into account knowledge about the biodegradability of a polymer in early stages of a product design process. Thus, it would be advantageous to provide a possibility to predict a biodegradability of a polymer accurately and in a computationally inexpensive manner.

### SUMMARY OF THE INVENTION

It is an object of the present invention to provide a method, an apparatus and a computer program product that allow for an accurate determination of a biodegradability of a polymer that is computationally inexpensive and can robustly be applied to new polymers. Moreover, it is further an object of the invention to provide a training method, a training apparatus and a computer program product that allow to provide a biodegradation model that is usable in the method, apparatus and computer program and that can be trained to provide a good determination accuracy by utilizing less computational resources.

In a first aspect of the present invention, a computer implemented method for determining a biodegradability usable for validating a biodegradation for a polymer is presented, wherein the method comprises i) providing a digital representation of the polymer indicative of or associated with physicochemical characteristics of the polymer, ii) providing a biodegradation habitat, wherein a biodegradation habitat is indicative of habitat descriptor values of habitat descriptors influencing a biodegradation of a polymer in the respective habitat, wherein the habitat descriptors are indicative of environ-mental characteristics of the habitat, iii) providing a biodegradation model based on the provided biodegradation habitat, wherein the biodegradation model is adapted to determine a biodegradability of a polymer in the respective biodegradation habitat, wherein the biodegradation model is a data driven model parametrized with respect to the biodegradation habitat such that it can determine a biodegradability of a polymer based on the physicochemical characteristics, and iv) determining the biodegradability of the polymer based on the provided biodegradation model and the digital representation of the polymer.

Since the biodegradation model is specifically adapted to determine a biodegradability of a polymer in a specifically provided biodegradation habitat characterized by respective habitat descriptor values influencing a biodegradability of a polymer in the respective habitat, the biodegradability of a polymer for the respective habitat can be determined very accurately. Moreover, since the biodegradation model has specifically been trained for one or more specific biodegradation habitats, less training data becomes necessary for the training and the biodegradation model becomes more flexible with respect to determining the biodegradation of new polymers not being part of the training data set. Thus, the method allows for an accurate determination of a biodegradability that is computationally inexpensive and can be applied flexible also to new polymers. Furthermore, currently utilized test methods for testing a biodegradability of a polymer are extremely time consuming and can take months or years to get results, whereas the above described method allows to provide results essentially immediately. Thus, not only the technical requirements for biodegradability determination can be reduced, but also the time required for designing a new biodegradable product can be considerably shortened. Moreover, providing an easy possibility for taking an accurate determination of the biodegradability of a product into account already during the design process allows to design the product such that polymer waste, in particular, in form of micro-plastic, can be avoided. In particular, it can be ensured that a polymer used in a product will biodegrade in a respectively expected environment, for example, in a marine habitat.

Moreover, since the physicochemical characteristics are utilized that contain physicochemical information of the polymer, e.g. quantum chemical information of the polymer like solubility in water and octanol or molar mass of the polymer, the training of a respective biodegradation model can be improved. In particular, utilizing the physicochemical characteristics allows training of such models with less training data, because some of the correlation information that needs to be learned is already presented to the model by using the physicochemical characteristics. This, further allows to save tests and experiment necessary for providing the training data set.

Development of new chemical products that are tailored to application requirements is a predominant problem in modern chemical industries. Recently, a further requirement is also raised, related to the environmental impact of the chemical product along its life cycle. One important aspect of the environmental impact is prevention of micro-plastics and bioaccumulation. Micro-plastics are an increasing problem and can be avoided if the polymeric material is biodegradable. To evaluate biodegradation currently a series of standardized tests, are used. For biodegradability, a variety of tests exists with specified conditions (e.g. ISO13432, ISO14852, ISO14855, ISO17556 and OECD 301). Standardized tests often strike a balance between a time-efficient testing (shortest 14 days, longest 24 months) and real-life conditions. In fact, higher temperatures than real conditions are often used to speed up the testing time. Companies developing new polymers need to invest significant resources in self-assessing product sustainability and in certification. The overall biodegradability assessment, including laboratory spaces and equipment, becomes costly and time consuming. Thus, there is a need to early identify the biodegradability of a new material in the development process. The proposed method of determining biodegradability as disclosed herein enables a faster and more efficient way of developing new materials. In an early phase, even before synthesis of the polymer, the biodegradability can be determined. This allows to determine whether the polymer is suited for market entry. This leads to a faster time to market. This also allows to reduce resource demands and waste production, because the polymer does not need to be synthesized to determine biodegradability. The proposed method provides a digital twin of measuring the biodegradability of a polymer.

Further, the standard measurements and tests for a biodegradability are often time consuming, for example, include waiting times of up to several months or even years. In particular when developing new polymers for respective applications these time consuming tests can strongly limit the development process. In this context the invention allows to provide results for a new polymer instantly strongly decreasing the time after which results are available.

Moreover, due to the incredibly high number of possible, often not even fully explored polymers, potentially suitable for a specific application, today a technical product engineer, given the technical task of finding a polymer that is not only suitable for a specific application, but also fulfills respective target properties, in particular, a target biodegradability, has to synthesize and test huge amounts of possible polymers, or go through huge datasets and libraries in which potential polymers are stored in order to find a respective polymer that might fit the application. Even when utilizing sophisticated design of experiment methods, still a very high number of possible polymers has to be synthesized and experimentally tested. In this context the above described method allows to assist a user, for instance, a technical product engineer, to find potentially suitable polymers automatically and much faster. In particular, by utilizing the above method the user only has to synthesize and test potentially suitable polymers for which it has been determined that it is very likely that they fulfill the respective target property, in particular, a target biodegradability. Accordingly, unnecessary synthesizing and testing of polymers can be avoided. Thus, the method allows a user to perform a technical task of finding a polymer suitable for a technical application faster and more efficient.

The method refers to a computer implemented method and thus can be performed by a general or dedicated computer adapted to perform the method, for instance, by executing a respective computer program. The method is adapted to determine, in particular, predict a biodegradability of a predetermined polymer. A biodegradable polymer refers to a polymer that can be degraded by biological processes, in particular, a biodegradable polymer can refer to a polymer that can be assimilated by bacteria and/or fungi to give environmentally friendly products, i.e. to decompose into non-polluting residuals, for example by produce mineralized carbon and/or biomass. Generally, the determined biodegradability can refer to any quantification of the biodegradability of a polymer. For example, the determined biodegradability can refer to only one value, for instance, a half-life of the polymer in a respective habitat, or can refer to more than one value, for instance, can refer to a degradation function with time of the polymer in a specific habitat. Preferably, the biodegradability refers to a value of the percentage of biodegradation after a predetermined timeframe. Generally, the biodegradability refers to a measure for a degradation, i.e. decomposition, of a polymer caused by biological processes, i.e. processes that include biological material, in particular, microorganisms taking part in the degradation process. Thus, the biodegradability does not refer to purely chemical degradation processes that do not include microorganism activity. The biodegradability is an intrinsic characteristic of a polymer. In this context, an intrinsic characteristic of a polymer refers to a property of the polymer that is caused by and thus reflects the nature of a polymer, i.e. its structure, composition, etc., with respect to a specific context. In particular, the biodegradability reflects the nature of the polymer when present in a specific biological active environment. For example, it is preferred that the biodegradability of the polymer refers to any one of a mineralization characteristic, a biotransformation characteristic and/or a decomposition of the polymer after a specific timeframe. Moreover, the biodegradation is a technical characteristic of a polymer, i.e. knowledge of the biodegradation of a polymer strongly influences the technical applicability and utilization of a polymer. The biodegradability is usable, in particular, for validating a biodegradation, i.e. a degradation characteristic, of a polymer, for example, for a specific biodegradation environment, i.e. biodegradation habitat. For example, the biodegradability can be compared with required biodegradabilities for a specific application and thus it can be determined if a respective polymer is suitable for a respective application.

Generally, the polymer can be any polymer. Preferably, the target polymer is a synthetic polymer. In an embodiment, a synthetic polymer may be a chemical compound which is produced by a chemical production from one or more starting material(s), such as monomers, and which comprises at least two monomer units. The monomer units may be regarded as subunits of the polymer. The polymer may be prepared from the monomers by commonly known polymerization techniques. The polymer may be produced from a single type of monomers or from different monomers. The polymer may be produced by a single polymerization technique or by a combination of different ones. The monomer units may be distributed randomly or may be present as blocks within the polymer. The polymer may be a linear polymer. The polymer may be a branched polymer. The polymer may be a crosslinked polymer. The polymer may be chemically modified after polymerization.

In a first step, the method comprises providing a digital representation of the polymer indicative of physicochemical characteristics of the polymer. In particular, the providing can refer to receiving the digital representation from an input of a user using, for instance, a respective input unit. Moreover, the providing can also refer to accessing a storage unit on which the digital representation is already stored. Further, the providing can also comprise receiving physicochemical characteristics, for instance, via a network connection from other sources and providing the received physicochemical characteristics as digital representation. Moreover, being indicative of or associated with physicochemical characteristics of a polymer is defined as allowing to access the information of the physicochemical characteristics. For example, the digital representation can directly comprise the physicochemical characteristics, for example, in form of values for respective quantities. However, the digital representation can also be a link to the respective physicochemical characteristics via which the physicochemical characteristics can be accessed, or the digital representation can refer to an identifier that is associated with the physicochemical characteristics and allows to utilize a respective look up storage in order to access the physicochemical characteristics. Moreover, the digital representation can also refer to information that allows to derive the physicochemical characteristics using one or more known relations. For example, a synthesis specification or a structural formula of a polymer can be utilized as digital representation allowing to derive, using known chemical and physical laws and relations, respective physicochemical characteristics.

Generally, throughout the following description referring a parameter or a characteristic comprises referring both to the respective quantity and also to a specific value of the quantity if not explicitly defined otherwise. For example, a parameter being a temperature always refers to the quantity being a temperature and also to a specific value of the temperature being set for the quantity. Since in most cases the explicit value of the parameter can be different for different embodiments and application cases the value is generally not mentioned. However, providing a parameter or characteristic generally means providing the quantity, e.g. the information that a value is a temperature, and also the value of the quantity or characteristic itself.

In particular, the physicochemical characteristics of a polymer can be quantified by polymer physicochemical parameters. Preferably, the digital representation is indicative of and/or comprises polymer physicochemical parameters, preferably, referring to polymer descriptors, wherein the polymer physicochemical parameters are indicative of the physicochemical characteristics of the polymer. In particular, the polymer physicochemical parameters are indicative of parameters quantifying the physicochemical characteristics of the polymer. In this context, the term “physicochemical characteristics” refers to physical and/or chemical characteristics of the polymer. However, the digital representation can also be provided such that it allows to derive physicochemical characteristics, for example, in form of the polymer descriptors, for instance, by providing a representation of the polymer for which respective physicochemical characteristics are already stored or can be determined, for instance, by respective polymer descriptor calculations. Preferably, the digital representation refers to at least one of a recipe, a structural formula, a brand name, an IUPAC name, a chemical identifier and a CAS number of the polymer.

In a preferred embodiment, the polymer physicochemical parameters are parameters quantifying the physicochemical characteristics of subgroups of the polymer. In this embodiment, the digital representation can also be provided such that it allows to derive the polymer physicochemical parameters by determining subgroups of the polymer and to determine the polymer physicochemical parameters based on physicochemical characteristics of the determined subgroups. Generally, a subgroup refers to a part of the polymer, wherein all subgroups of a polymer together form the polymer. For example, a subgroup can refer to a part of the polymer, wherein the subgroups are linked together successively along a chain or network to form the polymer. Preferably, the subgroups of the polymer refer to repeating units that describe a part of the polymer which when repeated produces the complete polymer chain. However, in some cases, a subgroup can also refer to a single part of the polymer that is not repeated. Moreover, it is preferred that the subgroups comprise parts that are repeated, for example, a subgroup of a polymer can comprise a repeating core also present in other subgroups and further additional parts that are not present in other subgroups. Preferably, the subgroups refer to at least one of polymerized monomers or oligomer fragments. More preferably, the subgroups refer to polymerized monomers. In this context, polymerized monomers refer to monomers after their polymerization sometimes also called “mer unit” or “mer”. In particular, polymerized monomers do not refer to monomers, i.e. raw materials, as present in a reaction mixture before polymerization, but refer to repeating units derived from monomers that have been changed during or after the polymerization. Thus, subgroup descriptors determined for polymerized monomers are different from subgroup descriptors determined for unreacted monomers before polymerization. It has been found by the inventors that in particular the polymerized monomers allow to determine polymer descriptors from the subgroup descriptors of the polymerized monomers that allow for an accurate determination of the biodegradability. In a preferred embodiment, the digital representation of the polymer comprises subgroups provided as molecular model which is indicative of its chemical structure of the subgroup after its polymerization. Even more preferably, the molecular model of a subgroup is determined in a way that is suited for quantum chemical computations regarding a number and type of atoms and their connectivity that is representative of the properties of the subgroup within the polymer. Moreover, additionally an alternatively to a molecular model of a subgroup treating the subgroup as a monomer structure, also a molecular model referring to an oligomer model can be utilized that takes into account effects of neighbouring molecular structures of the subgroup in the polymer.

Generally, if the digital representation of the polymer does not directly comprise the polymer physicochemical parameters, it is preferred that the polymer physicochemical parameters are determined by determining the subgroups of the polymer. For example, respective subgroups of the polymer can be determined utilizing known methods. However, it is preferred that the determination of the subgroups of the polymer is performed in accordance with later described embodiments of the invention. In particular, it is preferred that the subgroups are determined such that between atoms of different subgroups in the polymer the bond is as least polarized as possible and, preferably, with a bond order as small as possible (e.g. a CC single bond). Additionally, it is preferred that the subgroups representing a polymer comprise the same number of active non-hydrogen-atoms then the polymer. Besides the active atoms, a subgroup can also contain further atoms, which can be ignored during computing the descriptors of the subgroup. Further, it is preferred that the subgroups are determined in a way that polymers comprising parts, which were built up with different polymerization techniques, are well covered and fulfill the foresaid conditions. An example is a polyether used as ingredient for a polyurethane. Generally, a database or archive with a plurality of reactions between polymer parts can be generated and the subgroups can be derived from the respective structure of the reactions. For example, specific chemical languages like SMILES and SMARTS can be utilized to easily derive the subgroup of a polymer. For example, a database of reaction SMARTS can be generated and then based on the polymerization of the respective polymer a corresponding reaction SMARTS can be selected. From the selected reaction SMARTS then the SMILES of monomers of the polymer are directly derivable and, for example, RDkit can be used to determine from the SMILES of the monomers the SMILES, i.e. the number and connectivity of the atoms, of the subgroups.

The determined subgroups of the polymer are associated with subgroup physicochemical parameters quantifying physicochemical characteristics of the subgroups in the polymer, preferably, also the subgroup physicochemical parameters refer to subgroup descriptors. In particular, it is preferred that if the polymer physicochemical parameters are not directly provided by the digital representation, the polymer physicochemical parameters are determined by determining a respective subgroup physicochemical parameter for each of the subgroups and to determine the polymer physicochemical parameters based on the subgroup physicochemical parameters of the subgroups, for instance, by averaging. Thus, the method preferably comprises first providing or determining for the polymer the subgroups from the digital representation of the polymer, then to determine or provide the subgroup physicochemical parameters, i.e. values of the parameters quantifying the physicochemical characteristics, of the subgroups, and then to determine the polymer physicochemical parameters based on the subgroup physicochemical parameters of each polymer.

Preferably, the polymer physicochemical parameters refer to polymer descriptors referring to at least one of constitutional descriptors, count descriptors, list of structural fragments, fingerprints, graph invariants, 3D-descriptors and/or higher dimensional descriptors that are indicative of parameters quantifying physicochemical characteristics of the polymer. In a preferred embodiment the polymer descriptors refer to 3D descriptors, in particular, quantum chemical descriptors. Moreover, the inventors have found that in particular a molar mass describes the biodegradation of a polymer very accurately. Thus, it is in particular preferred that the physicochemical parameters comprise a molar mass of the polymer. Generally, the polymer physicochemical parameters can be derived from the subgroup physicochemical parameters, thus, also the subgroup physicochemical parameters can refer to the same descriptors as stated above. However, the physicochemical parameters can also be derived without utilizing subgroups, for instance, by quantum chemical simulations of the whole polymer. In the following the possible physicochemical parameters are defined in more detail. Also in these cases the defined physicochemical parameters can refer directly to the polymer physicochemical parameters or, optionally, to the subgroup physicochemical parameters.

A constitutional descriptor can refer to any of a potential, average molecular weight, polydispersity, charge, spin, boiling point, melting point, enthalpy of fusion, dissociation constant, Hansen parameter, protic, polar and dispersive contributions, Abraham parameter, retention index, TPSA, receptor binding constant, Michaelis-Menten constant, Inhibitor constant, Mutagenicity, LD50, bioconcentration, toxicity, biodegradation profile and viscosity.

A count descriptor can refer to any of a sum of atomic electro negativities, a sum of atomic polarizabilities, an amount of ingredients, a ratio of amounts of ingredients, a number of atoms and non H-atoms, a number of H, B, C, N, O, P, S, Hal and heavy atoms, a number of H-donor and H-acceptor atoms, a number of bonds, non-H or multiple bonds, a number of double, triple and aromatic bonds, a number of functional groups, a ratio of functional groups, a sum of bond orders, an aromatic ratio, a number of rings or circuits, a number of unpaired electrons, a number of rotatable bonds, rotatable bond fractions, and a number of conformers.

Polymer descriptors referring to a list of structural fragment descriptors can refer to at least one of a list of molecular fractions, a list of functional groups, a list of bonds, and a list of atoms. Fingerprint descriptors comprise preferably, at least one of MACCS keys, preferably, in bit format or total amount format, Morgan and other circular fingerprints, preferably, in bit format or total amount format, topological torsion, atom pairs, infrared and related spectra, fingerprint count, PubChem fingerprint, substructure fingerprint, and Klekota-Roth fingerprint. Graph invariants/topological indices descriptors comprise preferably at least one of topostructural indices and topochemical indices.

In a preferred embodiment the polymer physicochemical parameters are 3D descriptors comprising at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and non-polar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the polymer physicochemical parameters refer to 3D descriptors comprising at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or nonpolar surface area, excitation energies and intensities, infrared and/or UV absorption bands, reactivity measurements, particle charges and/or charge surface areas. A preferably utilized higher dimensional descriptor can comprise at least one of a conformational partition function, solubility, vapor pressure, activity coefficient, diffusion coefficient, partition coefficient, interfacial activity, rotational constant, moment of inertia, radius of gyration, compositional drift of polymer, density, viscosity, conformer weighted volume and area, conformer weighted H-donor, H-acceptor, protic, polar and/or non-polar surface area, charge distribution, conformational dipole moment and molecular refraction. Preferably higher dimensional descriptors are utilized that comprise at least one of solubilities, vapor pressure and activity coefficients, interfacial activity, conformer weighted H-donor, H-acceptor, protic, polar and non-polar surface area, and charge distribution.

The method further comprises providing a biodegradation habitat, wherein a biodegradation habitat is indicative of habitat descriptor values of habitat descriptors influencing a biodegradation of a polymer in the respective habitat. In particular, the providing can refer to receiving the biodegradation habitat from an input of a user using, for instance, a respective input unit. Moreover, the providing can also refer to accessing a storage unit on which the biodegradation habitat is already stored. Furthermore, the providing can also refer to a presetting of a biodegradability habitat. For example, if the method is utilized in a very specific context that is only sensible with one specific biodegradation habitat, the respective biodegradation habitat can be preset and thus has not to be provided as specific input. Further, the providing can also comprise receiving directly the habitat descriptor values of the habitat descriptors, for instance, via a network connection, from other sources and providing the received habitat descriptor values of habitat descriptors as biodegradation habitat. The provided biodegradation habitat can refer to a general habitat, for instance, can refer to a waste water habitat, wherein respective habitat descriptor values for the habitat descriptors for this habitat are then already stored on a respective storage which can be accessed. However, the provided biodegradation habitat can also directly comprise the respective habitat descriptor values for the biodegradation habitat to provide a further specification of the biodegradation habitat, for example marine benthic. Moreover, the providing of a biodegradation habitat can include providing a digital representation of the biodegradation habitat, wherein the digital representation can then be indicative of respective habitat descriptor values of habitat descriptors influencing a biodegradation of a polymer in the respective habitat.

Generally, the habitat descriptors are indicative of environmental characteristics of the habitat. In particular, the environmental characteristics of a biodegradation habitat can influence a biological activity in the respective habitat, for example, can influence a presence, growth or absence of specific microorganisms. Thus, the environmental characteristics defined by the habitat descriptors indirectly also influence the biodegradation of a polymer in the respective habitat. For example, if a polymer is biodegradable by a specific microorganism that needs a specific salt concentration, the polymer will biodegrade fast in a habitat providing such a salt concentration, like a marine habitat, but will biodegrade much slower in a habitat with not the right salt concentration, like waste water. Again, being indicative of or associated with habitat descriptors of a habitat is defined as allowing to access the information of the habitat descriptors. For example, the habitat can directly comprise the habitat descriptors, for example, in form of values for respective quantities. However, the habitat can also be a link to the respective habitat descriptors via which the habitat descriptors can be accessed, or the habitat can refer to an identifier that is associated with the habitat descriptors and allows to utilize a respective look up storage in order to access the habitat descriptors. Moreover, the habitat can also refer to information that allows to derive the habitat descriptors using one or more known relations. For example, a geolocation of an environment can be utilized together with the habitat allowing to derive, using knowledge on a respective geolocation, to derive respective habitat descriptors.

Preferably, the biodegradation habitat refers to any one of a marine habitat, a waste water habitat, a limnic habitat, an anaerobic habitat, a compost habitat or a soil habitat. In a preferred embodiment, the biodegradation habitat refers to a marine habitat and wherein the habitat descriptors refer to at least one of a salt concentration, a sedimentation type, oxygen level, location, sample depth, a water temperature, a nutrient concentration, for example, a nitrogen, phosphate, potassium, and/or dissolved organic carbon concentration, a pH value, an environmental type, oxygen content, and a microbial community. In a further preferred embodiment, the biodegradation habitat refers to a limnic habitat and wherein the habitat descriptors refer to at least one of a salt concentration, a sedimentation type, oxygen level, location, sample depth a water temperature, a nutrient concentration, a pH value, an environmental type and a microbial community. In a further preferred embodiment, the biodegradation habitat refers to waste water and the habitat descriptors refer to at least one of a water temperature, a microbial community, a sludge concentration, a nutrient concentration, a pH value, a test duration, a solid content, and an enzyme environment. Moreover, in this habitat the sludge can also be a separate habitat. Thus, in an embodiment the habitat can also be a sludge habitat, for example, as the aerobic part of a waste water treatment plant and the habitat descriptors refer to at least one of a solid content, pH, nutrient content, heavy metal content, microbial community. In a further preferred embodiment, the biodegradation habitat refers to soil and the habitat descriptors refer to at least one of a temperature, composition, for example, a sand and/or clay content, a pH value, a moisture content, a nutrient concentration, a microbial community, a nitrogen content, a water holding capacity and an enzyme environment. In a further preferred embodiment, the biodegradation habitat refers to compost and the habitat descriptors refer to at least one of a temperature, compost activity, a pH value, a moisture content, humidity, compost maturity, compost composition, compost origin, a nutrient concentration, a microbial community, a solid content, a water holding capacity, and an enzyme environment. Generally, the habitat can also refer to a habitat of a standard test utilized for determining biodegradability of a polymer. For example, standard tests as defined by ISO13432, ISO14852, ISO14855, ISO17556 and OECD 301 also define a specific habitat in which the biodegradation takes place. Thus the providing of the biodegradation habitat can also comprise providing, for instance, selecting via a user input, one of the standard tests, wherein the habitat descriptors then refer to the specific characteristics of the test, i.e. of the test environment and thus test habitat. Moreover, the habitat can also be defined by the biodegradation of a reference polymer or other reference chemical. In this case the habitat can be provided by providing the reference and its biodegradation. In this case the reference and its biodegradation are indicative of the habitat descriptors.

The method further comprises providing a biodegradation model based on the provided biodegradation habitat. In particular, it is preferred that the providing of the biodegradation model refers to a selecting of a biodegradation model based on the provided biodegradation habitat. For example, a plurality of biodegradation models can be stored on a biodegradation storage, wherein each biodegradation model has been trained for one biodegradation habitat, in particular, for different values or value ranges of habitat descriptor values of a biodegradation habitat. Based on the provided biodegradation habitat indicative of the habitat descriptor values, a respective suitable biodegradation model can then be selected from the plurality of biodegradation models. For example, a biodegradation model is suitable if the indicated habitat descriptor values fall within the ranges of the habitat descriptor values for which the biodegradation model has been trained. For example, a respective lookup table can be provided that allows for an easy comparison between the indicated habitat descriptor values and the descriptor value ranges for which the biodegradation models stored on the storage have been trained such that directly a suitable biodegradation model can be selected. However, in another embodiment the providing of a biodegradation model based on the provided biodegradation habitat can also refer to a user selection of the biodegradation model. For instance, the user can be provided with a preselection of biodegradation models that refer to the provided biodegradation habitat and then be allowed to select the respective biodegradation model that should be utilized. Generally, the possible stored biodegradation models refer to biodegradation models that have already been parameterized based on a respective training data set for one or more habitats. Since the training data sets utilized for parameterizing a biodegradation model are historical data, as described in more detail below, the biodegradation models can be trained and thus generated at any time before the determination of a specific biodegradation for a specific polymer, and after the training be stored on a respective database. However, the training and thus the generation of a biodegradation model can of course also be performed at the time that it is determined that a specific biodegradation model, for instance, for a specific habitat, is needed.

The provided biodegradation model is then adapted to determine a biodegradability of a polymer in the respective biodegradation habitat. In particular, the biodegradation model is a data driven model that is parameterized with respect to the biodegradation habitat such that it can determine the biodegradability of a polymer based on the physicochemical characteristics, preferably, based on the polymer physicochemical parameters quantifying the physicochemical characteristics, indicated by the digital representation. The term “such that” is to be interpreted here that the parameterization adapts and thus enables the biodegradation model to provide the biodegradability with respect to a habitat when provided with polymer physicochemical parameters as input. For example, the biodegradation model relates polymer physicochemical parameters of historic digital representations of synthesis specification and historic digital representations of habitats to a biodegradability. This allows that, based on a target biodegradability, a digital representation of the synthesis specification may be determined. The term “data driven” is used here to emphasize that the model is mainly based on respective data input and not, for instance, on intuition, personal experience or knowledge. Preferably, the biodegradation model refers to a machine learning based model that is based on known machine learning algorithms, like neural networks, regression models, classification algorithms, etc. It has been found that for most applications in this context, in particular, regression models based on Linear Regression, Random Forests, Boosted Trees, Lasso, Ridge Regression and MARS algorithms are suitable, whereas for classification models, in particular, Random Forests, Logistic Regression and SVM algorithms are suitable. Preferably, the biodegradation model is based on a neural network algorithms. Generally, the biodegradation model is parameterized during a training process in which physicochemical characteristics, preferably, polymer descriptors derived from parameters quantifying the physicochemical characteristics of the polymer, are utilized together with corresponding biodegradabilities for specific biodegradation habitats. Based on such a training data set that is specific for a biodegradation habitat, for instance, for specific habitat descriptor value ranges and/or values, the respective parameters of the data driven model can be determined utilizing known training methods such that the biodegradation model is also able to determine a biodegradation of polymers that are not part of the training data set.

Moreover, in a preferred embodiment, the biodegradation model can also be adapted to determine the biodegradation for a polymer further based on habitat descriptor values as input. In particular, the biodegradation model can be trained by utilizing a training data set comprising polymer physicochemical parameters of polymers and associated biodegradabilities for a specific habitat, as described above, leading to a biodegradation model that indirectly takes the specific habitat into account. However, the training data set can optionally also comprise specific habitat descriptor values of a respective habitat. In this case, the biodegradation model can be trained such that in addition to the polymer physicochemical parameters also habitat descriptor values can be provided as input, wherein the biodegradation model then determines the biodegradability further based on the habitat descriptor values. This has the advantage that the biodegradability can be determined even more accurately, in particular, in cases in which the biodegradation strongly depends on the specific habitat descriptor values of the habitat. For example, in a marine habitat a temperature or salt concentration can strongly deviate for different regions of the world, wherein for some polymers this can also lead to different biodegradabilities. Thus, for such cases it can be advantageous to directly provide the habitat descriptor values as input to the biodegradation model. However, it is also possible instead of providing the habitat descriptor values as input to biodegradation model, to train two different biodegradation models and indirectly treat the different regions as different habitats.

Further, the method comprises determining the biodegradability of the polymer based on the provided biodegradation model and the digital representation of the polymer. In particular, if the digital representation of the polymer comprises the polymer physicochemical parameters, the polymer physicochemical parameters are provided as input to the biodegradation model, wherein the biodegradation model then provides the determined biodegradability as output. If the digital representation does not directly comprise the polymer physicochemical parameters, the determining of the biodegradability can comprise also determining firstly the polymer physicochemical parameters, for instance, as described above. The such determined polymer physicochemical parameters can then be provided to the biodegradation model as input. The determined biodegradability application property can then be provided, for instance, to an output unit or to a computing unit for further processing. Preferably, the determined biodegradation is provided such that the biodegradation is usable for validation of the polymer, in particular, with respect to an intended application. Preferably, the determined biodegradability is provided such that it can be validated whether the biodegradation of the polymer fulfils a predetermined target. Even more preferably, the providing comprises the validation of the polymer based on the determined biodegradation. In an embodiment, based on the validation a specification of the polymer can be provided together with the biodegradation.

Preferably, the providing of the biodegradability leads to a further processing utilizing the determined biodegradability. In such a case, the providing as individual step can be omitted and replaced by the processing of the determined biodegradability.

The determination of the biodegradability utilizing the biodegradation model can be regarded as a virtual measurement of the biodegradability. In particular, the biodegradation model is based on measurement data, for example, measured biodegradabilities of polymers utilized for the training of the biodegradation model. Thus, the biodegradation model comprises the information provided by these previous measurements. Moreover, the physicochemical parameters can in some cases also refer to measured characteristics of the polymer. Accordingly, also the determined biodegradability of new polymer determined utilizing the biodegradation model can be regarded as being based at least partly on measurement results.

Preferably, the processing of the biodegradability comprises determining control signals for controlling a production process based on the determined estimated biodegradability. The production process can refer to a production process of the polymer or can refer to a biodegradation process of a product in which the polymer is utilized. For example, if the determined biodegradability indicates that polymer will biodegrade in a specific environment, i.e. habitat, in a suitable fast manner, the generation of the controlling signals can comprise generating controlling signals for controlling a waste management facility to provide this habitat, for instance, by providing a respective temperature. In a preferred embodiment the control signal is indicative of a machine executable synthesis specification of the polymer, in particular, when a comparison indicates that the determined biodegradability of the polymer lies within a predetermined range around a provided target biodegradability.

Moreover, the process of processing the biodegradability can also refer to a step of selecting one or more polymers based on respectively determined biodegradabilities. For example, if for a plurality of potential polymers respective biodegradabilities have been determined, the selecting can comprise comparing the biodegradabilities of the different polymers to predetermined selection criteria and select the polymers for which the determined biodegradabilities fulfill these criteria. In particular, in an embodiment the method comprises receiving a target biodegradability for a polymer and comparing the received target biodegradability with a determined biodegradability and providing depending on the comparison a control signal. The control signal can refer to any signal that allows for a further control of a technical system. For example, the control signal can be adapted to control an interface for providing the result of the comparison on the interface.

Preferably, the processing of the biodegradation is a validation of the biodegradation. In a preferred embodiment the comparison refers to a validation of the target biodegradability, wherein the validation is positive if the determined biodegradability falls within a predetermined range around the target biodegradability. In this case the control signal can be adapted to simply control a user interface to provide an indication of a positive or negative validation result. However, preferably, the control signal refers to a recipe, i.e. synthesis specification, of the one or more polymers which fulfill the specified target biodegradability, i.e. which are validated positively. A recipe, i.e. synthesis specification, is generally defined as an instruction on how a polymer can be synthesized. In particular, the recipe comprises the starting substances and the respective parameters for polymerization from the starting substances. Preferably, the control signals comprise a recipe in a form that directly allows an automatic controlling of respective industrial systems or labor equipment for producing the polymer. In particular, it is preferred that the control signal is indicative of a machine executable synthesis specification of the polymer, when the result of the comparison refers to the determined biodegradability being within a predetermined range around the target biodegradability.

In an embodiment, the method comprises validating the polymer based on the determined biodegradation. In particular, the validation can be performed as described above. Moreover, in a preferred embodiment, the validation comprised determining a biodegradation classification for the polymer based on the determined biodegradation. For example, the classification can refer to classifying whether the polymer is biodegradable or not. Moreover, the classification can refer to determining to which a plurality, for instance, three, biodegradation classes a polymer belongs. Preferably, the classification is based on one or more predetermined biodegradation thresholds determining the class of a polymer. For example, a polymer with a determined biodegradation of 0% to 25% in a predetermined time in a habitat can be classified as not-biodegradable, a polymer with a determined biodegradation of over 25% and under 60% in a predetermined time in a habitat can be classified as a medium biodegradable polymer and a polymer with a determined biodegradation of over 60% in a predetermined time in a habitat can be classified as highly biodegradable.

In a preferred embodiment the method further comprises providing as digital representation of the polymer a synthesis specification and determining the physicochemical characteristics, for example, in form of polymer physicochemical parameters, preferably, as polymer descriptors, from the synthesis specification. In particular, the synthesis specification, i.e. recipe, comprises information on the polymer synthesis of the polymer, for instance, on the starting substance and process by which respective starting substances are covalently bonded to form the polymer chain or network of the polymer. The method then comprises determining physicochemical characteristics, for instance, the polymer descriptors, from the synthesis specification. Optionally, from a synthesis specification the subgroups can be determined and the polymer physicochemical parameters can then be determined based on subgroup physicochemical parameters of the subgroups, for instance, from a database or utilizing known physicochemical parameter determination algorithms. In a preferred embodiment, further from the synthesis specification utilized catalysts and/or non-reactive process ingredients are determined. In this case it is preferred that this information is also utilized together with the physicochemical characteristics, in particular, the polymer physicochemical parameters, by the biodegradation model for determining the biodegradability. Preferably, physicochemical parameters are also determined for the catalysts and/or nonreactive process ingredients and the respective physicochemical parameters are also used for determining the physicochemical parameters of the polymer. Preferably, the physicochemical parameter for the catalysts and/or non-reactive process ingredients refers to an amount of the respective ingredient, for example, a molar mass, a molar percentage, etc. and is taken into account for determining a polymer physicochemical parameter for the polymer.

In a preferred embodiment, the determining of polymer physicochemical parameters from the synthesis specification comprises identifying types and amounts of subgroups based on the synthesis specification, for instance, as physicochemical parameters of the subgroups, and determining the polymer physicochemical parameters based on the identified types and amounts of subgroups. Generally, the types of subgroups can refer to predetermined types or classes that are associated with specific physicochemical characteristics, i.e. physicochemical parameters, of the subgroups, and thus with specific physicochemical characteristics of a polymer comprising these subgroups. However, since the general physicochemical characteristics of a polymer and hence the polymer physicochemical parameters can also depend on the amount of a subgroup present in the polymer that amount can also be taken into account. In a preferred embodiment the determination of the type and amount of subgroups takes into account information provided by the synthesis specification indicative of the type of polymerization. The information on the type of polymerization that can be utilized can refer, for instance, to whether the polymerization refers to a polycondensation, polyaddition, radical polymerization, cationic polymerization, anionic polymerization, or coordinative chain-polymerization. Preferably, for each type of polymerization rules are predetermined that can be applied to determine the subgroups of the polymer. For example, rules can be predetermined that determined which functional groups of monomers in the synthesis specification react with which prioritization to which functional groups of the synthesized polymer. The rules can be based, for instance, on kinetic considerations. Based on the number and type of polymerized functional groups the subgroups can be determined and a number and type of the subgroups can be calculated.

In an embodiment the determination of the amount of subgroups comprises determining the amount of at least one of, amide, ester, thioester, carbonate, ether, amine, urea, urethane, thiourethane, isocyanurate, biuret, allophanate, acetal, Michal-adduct, radically polymerized double bond, siloxane, silane, silazane, phosphazene groups as well as residual amine, aldehyde, ketone, epoxide, aziridine, isocyanate, alcohol, thiol, carboxylic acid, acyl halogenide, α,β-unsaturated carbonyl groups, α,β-unsaturated carboxyl and double bond groups in the polymer based on the synthesis specification.

In an embodiment, the method further comprises providing a biodegradation test method, wherein the provided biodegradation test method is indicative of a standardised biodegradation test method for determining experimentally a biodegradation of a chemical, wherein the biodegradation model is further provided based on the provided biodegradation test method. Generally, a plurality of standardized biodegradation test methods exists for testing a biodegradation of a chemical. For example, such test methods can be found in DIN or ISO norms. Further providing a biodegradation test method and providing a biodegradation model that has been trained based on the provided biodegradation test method allows to determine a biodegradation that is easily comparable, for instance, with respectively measured biodegradations utilizing the respective test method. In particular, for this embodiment it is preferred that the biodegradation models are trained based on data sets in which the test method based on which the biodegradation has been determined is clearly specified such that a biodegradation model can be trained specifically for one or more test methods.

In an embodiment, further a target application of the polymer is provided referring to an intended application of the polymer, wherein the biodegradation habitat is provided based of the target application. A target application of a polymer can refer, for instance, to an intended application context of the polymer, for example, if it is intended to utilize the polymer as a coating, in personal care products, in a washing detergent, in a lubricant, in agriculture or in a packaging of a product. Such target applications indicate specific biodegradation habitats. For example, for a packaging of a product it could be interesting if a polymer biodegrades in a compost. In another example, if the target application refers to utilizing the polymer in personal care products, it is very likely that the polymer will sooner or later be found in a water environment. Thus, a respective target application can be indicative for a respective biodegradation habitat. In this context, a predetermined list can be provided on a storage on which respective target applications and corresponding biodegradation habitats are stored. A target application for a polymer can then be provided, for instance, by providing the list of target applications to a user and allowing the user to select a respective target application, wherein a respective target application is connected to one or more biodegradation habitats. A biodegradability can then be determined for each of the biodegradation habitats to which the target application is connected or again a user can select a respective biodegradation habitat connected with the target application. Additionally or alternatively, information indicative of an intended end-of-life treatment of the polymer can be provided. For example, an end-of-life treatment can be indicative of, whether the polymer is intended to biodegrade in a specific environment, or should be subjected to a specific treatment, for example, in a bioreactor. Thus, also the information of the intended end-of life treatment can be utilized to determine a biodegradation habitat for the polymer, as described above.

In an embodiment, further information indicative of an accessible surface area of the polymer in its intended form is provided, wherein the biodegradation model is further trained to determine a biodegradability based on the accessible surface area, and wherein the method further comprises determining the biodegradability further on the accessible surface area. For example, the information can refer to whether the intended product is provided in a solid, pulverized, foamy, pelletized, or any other form. Preferably, the information is indicative of a surface area of the product per mass or a geometry of a smallest independent part of the product. Generally, although the biodegradability of a polymer is an intrinsic characteristic of the polymer, the exact timing of the biodegradability of a product comprising the polymer can also depend on the surface area that can be accessed, for instance, by microbial components of the habitat responsible for the biodegradation. Thus, further determining the biodegradability based on a surface area of a product comprising the polymer allows to increase the accuracy in the prediction of the biodegradability of the final product.

In an embodiment, habitat descriptor values for the habitat descriptors are stored associated with respective geolocations, wherein the providing of a biodegradation habitat refers to providing a geolocation of the habitat and retrieving the habitat descriptor values for the geolocation from storage. Geolocations can refer, for instance, to coordinates, or other regional identifications. For example, a geolocation can refer to the name of a city, country, country region, sea region, geographical feature, etc. Based on such geolocations, respective habitat descriptors, for instance, average values, or minimal and maximal values of the habitat descriptors, can be stored. Thus, by providing the geolocation, the respective habitat descriptor values for this geolocation can be provided. This has the advantage that an exact habitat or exact habitat descriptor values for a region do not have to be known to a user. Thus, the user can simply provide a location for which it is expected that the polymer might biodegrade in this region can be provided.

In an embodiment, polymer physicochemical parameters indicated by the digital representation of the polymer refer at least to one of recipe parameters from polymer synthesis, constitutional descriptors, count descriptors, list of structural fragments, fingerprints, graph invariance, 3D-descriptors and/or higher dimensional descriptors that are indicative of a chemical nature of the polymer. Respective connections of the digital representation with polymer descriptors, for instance, calculated previously, or further information on the polymer, can be stored already and connected with the respective digital representation. For example, if the digital representation refers to a brand name, a respective structural formula, subgroups and/or subgroup physicochemical parameters or polymer physicochemical parameters corresponding to the brand name can be stored already, for example, on a storage of the brand name owner.

In a preferred embodiment, the polymer belongs to a polymer type being a least one of a polyalkoxylate, polycondensate, addition polymer, vinylic polymer, natural polymer, polymer dispersion, polymer foil, biopolymer, polysilicone, resin, rubber and polyketone, wherein the biodegradation model is specifically trained for the respective polymer type to which the polymer belongs. In particular, the training data for parameterizing the biodegradation model comprises polymers of the respective polymer type. However, the biodegradation model can also be parameterized with training data of polymers from more than one polymer type.

In a preferred embodiment, the polymer belongs to polyalkolates and the habitat is a waste water habitat, in particular, a sludge habitat. Moreover, it is preferred in this embodiment that the physicochemical characteristics comprise at least one of a molar mass, an ingredient, a chemical moiety, solubility in water and a partition coefficient, more preferably, the physicochemical characteristics comprise a molar mass, even more preferably, comprise a molar mass, an ingredient, even more preferably, comprise a molar mass, an ingredient, and a partition coefficient.

In a preferred embodiment, the polymer belongs to polycondensates, preferably, polyesters, polyamides and phenoplasts, and the habitat is a waste water habitat, in particular, a sludge habitat, or soil habitat. Moreover, it is preferred in this embodiment that the physicochemical characteristics comprise at least one of a molar mass, an ingredient, chemical moiety, solubility in water, a partition coefficient, a measure for stability against hydrolysis, and degree of crystallinity, more preferably, the physicochemical characteristics comprise a molar mass and an ingredient, even more preferably, comprise a molar mass, an ingredient and a chemical moiety, even more preferably, comprise a molar mass, an ingredient, chemical moiety, and a degree of crystallinity.

In a preferred embodiment, the polymer belongs to addition polymers, preferably, polyurethanes and polyureas, and the habitat is a soil habitat or marine habitat. Moreover, it is preferred in this embodiment that the physicochemical characteristics comprise at least one of an ingredient, a chemical moiety, a ratio of chemical moieties, a ratio of ingredients, and a degree of crystallinity, more preferably, the physicochemical characteristics comprise an ingredient, even more preferably, comprise an ingredient and a ratio of chemical moieties, even more preferably, comprise an ingredient, a ratio of chemical moieties, a ratio of ingredients.

In a preferred embodiment, the polymer belongs to the vinylic polymers, preferably, polyvinyls, polyacrylates, polystryrenes polyvinylethers, and polyvinylalcohols and the habitat is a waste water habitat, in particular, a sludge habitat, or a soil habitat. Moreover, it is preferred in this embodiment that the physicochemical characteristics comprise at least one of a molar mass, an ingredient, a chemical moiety, solubility in water, a partition coefficient, more preferably, the physicochemical characteristics comprise a molar mass, even more preferably, comprise a molar mass, and an ingredient, even more preferably, comprise a molar mass, an ingredient and a chemical moiety.

In a preferred embodiment, the polymer belongs to natural polymers, preferably, polysaccharides, polynucleotides, lignin, suberin, cutin, cutan, melanin, natural rubber and polypeptides, and the habitat is a waste water habitat, in particular, a sludge habitat, or a soil habitat. Moreover, it is preferred in this embodiment that the physicochemical characteristics comprise at least one of a molar mass, a chemical moiety, and solubility in water and a partition coefficient, more preferably, the physicochemical characteristics comprise a chemical moiety, even more preferably, comprise a chemical moiety, and a molar mass.

In a preferred embodiment, the polymer belongs to polymer dispersions and the habitat is a marine habitat or soil habitat. Moreover, it is preferred in this embodiment that the physicochemical characteristics comprise at least one of an ingredient, a chemical moiety, a solubility in water and a particle size, more preferably, the physicochemical characteristics comprise an ingredient and a particle size, even more preferably, comprise an ingredient, a chemical moiety, and a particle size.

In a preferred embodiment, the polymer belongs to polymer foils and the habitat is a soil habitat or marine habitat. Moreover, it is preferred in this embodiment that the physicochemical characteristics comprise at least one of an ingredient, a molar mass, a chemical moiety, solubility in water, degree of crystallinity, and surface/volume ratio, more preferably, the physicochemical characteristics comprise an ingredient and surface/volume ratio, even more preferably, comprise an ingredient, a chemical moiety and a surface/volume ratio, even more preferably, comprise an ingredient, a chemical moiety, a degree of crystallinity, and surface/volume ratio.

In a preferred embodiment, the polymer belongs to polysilicones and the habitat is a soil habitat, waste water habitat, in particular, sludge habitat, or marine habitat. Moreover, it is preferred in this embodiment that the physicochemical characteristics comprise at least one of an ingredient, a molar mass, a chemical moiety, solubility in water, a partition coefficient, and surface/volume ratio, more preferably, the physicochemical characteristics comprise a molar mass, even more preferably, comprise a molar mass and an ingredient, even more preferably, comprise a molar mass, an ingredient, and a partition coefficient.

In a preferred embodiment, the polymer belongs to the resins and the habitat is a soil habitat or marine habitat. Moreover, it is preferred in this embodiment that the physicochemical characteristics comprise at least one of an ingredient, a molar mass, a chemical moiety, solubility in water, degree of crystallinity, and surface/volume ratio, more preferably, the physicochemical characteristics comprise a molar mass, even more preferably, comprise a molar mass and an ingredient, even more preferably, comprise a molar mass, an ingredient, and surface/volume ratio.

In a preferred embodiment, the polymer belongs to the rubbers and the habitat is a soil habitat or marine habitat. Moreover, it is preferred in this embodiment that the physicochemical characteristics comprise at least one of an ingredient, a chemical moiety, solubility in water, degree of crystallinity, and surface/volume ratio, more preferably, the physicochemical characteristics comprise an ingredient, even more preferably, comprise an ingredient and a surface/volume ratio, even more preferably, comprise an ingredient, a chemical moiety, degree of crystallinity, and surface/volume ratio.

In a preferred embodiment, the physicochemical characteristics comprise at least one of a molar mass, a chemical moiety, solubility in water and/or in octanol, a degree of crystallinity, and a surface/volume ratio. More preferably, the physicochemical characteristics comprise a chemical moiety, even more preferably, comprise a molar mass, a chemical moiety, and a solubility in water. For the polymer belonging to polyalkoxylates, polycondensates, vinylic polymers, or polyslicones it is preferred that the physicochemical characteristics comprise further at least one of a partition coefficient and an ingredient. For the polymer belonging to polycondensates, addition polymers, polymer foils, resins, rubbers, or polyketones it is preferred that the physicochemical characteristics comprise further at least one of a degree of crystallinity and a measure for stability against hydrolysis. For the polymer belonging to resins, rubbers, addition polymers or polysilicones, it is preferred that the physicochemical characteristics comprise further a surface/volume ratio.

In a further aspect, an interface method for providing an interface is presented, wherein the interface method comprises i) receiving as input a digital representation and a habitat via a user interface and providing the received digital representation and the habitat to a processor performing the method as described above, and ii) providing the determined biodegradability of the polymer to a user via a user interface as result, wherein the result is received from the processor performing the method as described above.

In a further aspect, a computer implemented training method for training a data driven based biodegradation model for parameterizing the biodegradation model is presented, wherein the training method comprises i) providing training data associated with a predetermined biodegradation habitat, wherein the training data comprises a) digital representations of a plurality of training polymers indicative of physicochemical characteristics for each of the training polymers, and b) a biodegradability for the respective biodegradation habitat associated with each training polymer, ii) providing a data driven based trainable biodegradation model, iii) training the provided data driven based biodegradation model based on the provided training data such that the trained biodegradation model is adapted to determine a biodegradation of a polymer based on a digital representation of the polymer, in particular, based on the physicochemical characteristics, preferably, polymer physicochemical parameters, more preferably, polymer descriptors, indicated by the digital representation, and iv) providing the trained biodegradation model.

In a further aspect, an apparatus for determining a biodegradability for a predetermined polymer is presented, wherein the apparatus comprises i) a digital representation providing unit for providing a digital representation of the polymer indicative of physicochemical characteristics of the polymer, ii) a habitat providing unit for providing a biodegradation habitat, wherein a biodegradation habitat is indicative of habitat descriptor values of habitat descriptors influencing a biodegradation of a polymer in the respective habitat, wherein the habitat descriptors are indicative of environmental characteristics of the habitat, iii) a model providing unit for providing a biodegradation model based on the provided biodegradation habitat, wherein the biodegradation model is adapted to determine a biodegradability of a polymer in the respective biodegradation habitat, wherein the biodegradation model is a data driven model parametrized with respect to the biodegradation habitat such that it determines a biodegradability of a polymer based on the physicochemical characteristics, and iv) a determining unit for determining the biodegradability of the polymer based on the selected biodegradation model and the digital representation of the polymer.

In a further aspect, an interface apparatus for providing an interface is presented, wherein the interface apparatus comprises i) an input interface unit for receiving as input a digital representation and a habitat via a user interface and for providing the received digital representation and the habitat to the apparatus as described above, and ii) a result interface for providing the determined biodegradability of the polymer to a user via a user interface as result, wherein the result is received from the apparatus as described above.

In a further aspect, a training apparatus for training a data driven based biodegradation model for parameterizing the biodegradation model is presented, wherein the training apparatus comprises i) a training data providing unit for providing training data associated with a predetermined biodegradation habitat, wherein the training data comprises a) digital representations of a plurality of training polymers indicative of physicochemical characteristics for each of the training polymers, and b) a biodegradability for the respective biodegradation habitat associated with each training polymer, ii) a trainable model providing unit for providing a data driven based trainable biodegradation model, iii) a training unit for training the provided data driven based biodegradation model based on the provided training data such that the trained biodegradation model is adapted to determine a biodegradation of a polymer based on a digital representation of the polymer, in particular, based on the physicochemical characteristics, preferably, the polymer physicochemical parameters indicated by the digital representation, and iii) a trained model providing unit for providing the trained biodegradation model.

In a further aspect of the invention a use of the method as described above is presented, wherein the method is used for determining a biodegradability for a predetermined polymer for any of the following i) polymers referring to polyesters, in particular, used for mulch film and packaging applications, e.g. aromatic aliphatic copolyesters, ii) polymers referring polyalkoxylates, in particular, used for home and personal care applications, iii) polymers referring to polyurethane dispersions, iv) polymers used for aroma applications, v) polymers used for paper coatings for packaging applications based on multilayer blends, and vi) polymers referring to polyurethane used for adhesives.

In a further aspect of the present invention, a system is presented, wherein the system comprises i) a control signal comprising a synthesis specification of a polymer indicating one or more ingredients for producing the polymer, wherein the control signals are generated according to the above described method, and ii) the one or more ingredients indicated by the synthesis specification in the control signal.

In a further aspect of the invention, a use of a control signal generated according to the above described method for controlling a production process, in particular, a production process comprising the production of a polymer is presented.

In a further aspect of the invention, a control signal is presented, wherein the control signal is generated according to the above described method. Preferably, the control signal comprises a machine executable synthesis specification for producing a target polymer.

In a further aspect, a computer program product for determining a biodegradability for a predetermined polymer is presented, wherein the computer program product comprises program code means for causing the apparatus as described above to execute the method as described above.

In a further aspect, a computer program product for training a biodegradation model is presented, wherein the computer program product comprises program code means for causing the training apparatus as described above to execute the training method as described above.

It shall be understood that the methods as described above, the apparatuses as described above and the computer program products as described above have similar and/or identical preferred embodiments, in particular, as defined in the dependent claims. Moreover, also the training method as described above, the training apparatus as described above and the training computer program product as described above have similar and/or identical preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system comprising an apparatus for determining a biodegradability of a polymer,

FIG. 2 shows schematically and exemplarily a flow chart of a method for determining a biodegradability of a polymer,

FIG. 3 shows schematically and exemplarily a flow chart of a method for training a biodegradation model for determining a biodegradability of a polymer,

FIG. 4 shows schematically and exemplarily a flow chart of an embodiment of a method for determining a biodegradability of a polymer,

FIG. 5 shows schematically and exemplarily an optional extension of a method for determining a biodegradability of a polymer,

FIGS. 6 to 8 show schematically and exemplarily a block diagram of a system architecture of a system and apparatus for determining a biodegradability of a polymer,

FIG. 9 shows schematically and exemplarily an output and input screen of an exemplary user interface, and

FIGS. 10 and 11 show schematically and exemplarily a further flow chart of preferred more detailed embodiments of a method for determining a biodegradability.

### DETAILED DESCRIPTION OF EMBODIMENTS

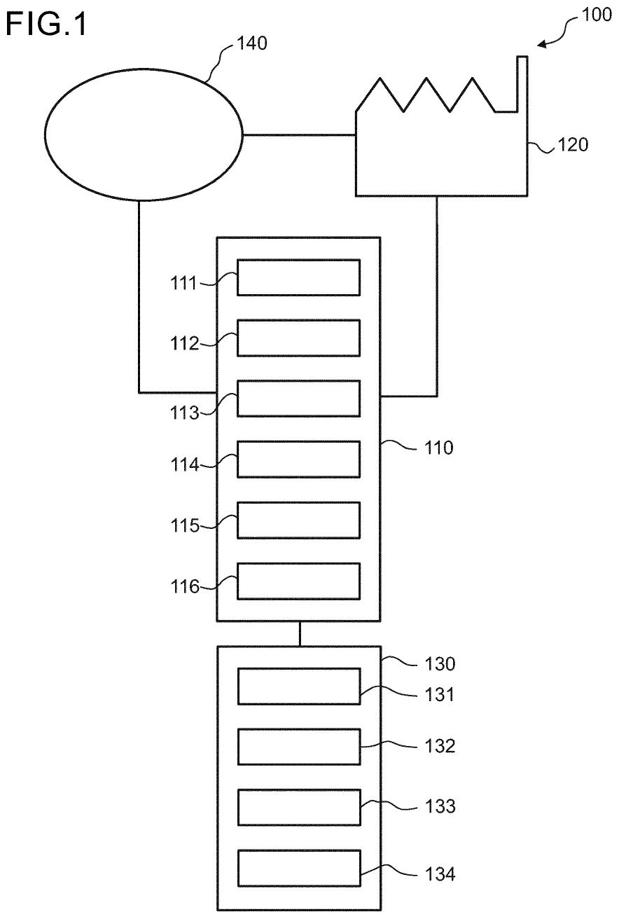

FIG. 1 shows schematically and exemplarily an embodiment of a system 100 comprising an apparatus 110 for determining a biodegradability of a polymer based on a digital representation of the polymer and a provided biodegradation habitat. Further, the system 100 comprises a training apparatus 130 for training a biodegradation model utilized in the apparatus 110, a database 140 on which determination results of biodegradabilities of polymers can be stored and a production system 120 for producing a product, in particular, comprising the polymer, that can be controlled utilizing the determined biodegradability.

The apparatus 110 comprises a digital representation providing unit 111, a habitat providing unit 112, a model providing unit 113, a determination unit 114 and optionally an output and/or control unit 115 that can be adapted to output the determined biodegradability and/or to provide control signals for controlling a production process of the production system 120 based on the determined biodegradability.

The digital representation providing unit 111 is adapted to provide a digital representation indicative of polymer physicochemical parameters, in particular, polymer descriptors, of a polymer for which a biodegradability should be determined. The digital representation providing unit 111 can refer, for instance, to an input unit into which a user can input the respective digital representation. Moreover, the digital representation providing unit 111 can refer to or be part of a user interface that allows the user to interact with the apparatus 110 and/or the database 140. However, the digital representation providing unit 111 can also refer to or be communicatively coupled with a storage unit on which the digital representation of the polymer is already stored. Generally, the digital representation can directly comprising the polymer physicochemical parameters that are indicative of parameters quantifying physicochemical characteristics of the respective polymer. However, instead of directly providing the polymer physicochemical parameters also a synthesis specification of the polymer can be provided. In this case, it is preferred that the digital representation providing unit 111 is further adapted to determine the polymer physicochemical parameters from the synthesis specification. In particular, it is preferred that the digital representation providing unit 111 is adapted to identify from the synthesis specification types and amounts of subgroups of the polymer and to determine the polymer physicochemical parameters based on the identified types and amounts of subgroups. In particular, the digital representation providing unit 111 can be adapted to determine for each identified subgroup respective subgroup physicochemical parameters, for instance, by accessing a database on which for a plurality of the most relevant subgroups respective physicochemical parameters are stored. The physicochemical parameters of the polymer can then be determined based on the subgroup physicochemical parameters of the subgroups and preferably, also on the determined amount and type of the subgroups, for example, by weighted averaging of the subgroup physicochemical parameters of the subgroups. The digital representation providing unit 111 is then adapted to provide the digital representation comprising the polymer physicochemical parameters, for instance, to the determination unit 114.

The habitat providing unit 112 is adapted to provide the biodegradation habitat. The habitat providing unit 112 can refer, for instance, to an input unit into which a user can input a respective biodegradation habitat. For example, a user interface can be provided that allows a user to select from a number of predetermined biodegradation habitats. In a preferred embodiment, the habitat providing unit can be communicatively coupled to or refer to a user interface that allows to indicate a geolocation, for instance, by marking a location on a map, by indicating coordinates, or providing a name of a region, for instance, a political or geological region, wherein the habitat providing unit can then be adapted to provide a biodegradation habitat based on the geolocation. For example, if the geolocation indicates a specific sea region like the North Sea or the Atlantic, the habitat providing unit can be adapted to determine as biodegradation habitat a marine habitat.

Generally, a biodegradation habitat is indicative of habitat descriptor values of habitat descriptors influencing a biodegradation of a polymer in the respective habitat. In particular, habitat descriptors are indicative of environmental characteristics of the habitat, for example, for a marine habitat a salt concentration can strongly influence the biodegradation of a polymer in the marine habitat. Generally, since the biodegradation is determined, the chemical influence of the habitat descriptors on the polymer is not important for this application. Thus, it is the influence of the habitat descriptors on the biology of the habitat, in particular, on the microbial population of the habitat, that indirectly influences the biodegradation.

The model providing unit 113 is adapted to provide a biodegradation model based on the provided biodegradation habitat. In particular, it is preferred that the model providing unit 113 is adapted to select the biodegradation model from a plurality of biodegradation models stored already on a database. For example, a biodegradation model can be trained with respect to training data corresponding to one or more specific biodegradation habitats. These specific biodegradation habitats can be defined with respect to habitat descriptor values or value ranges that define for which biodegradation habitat the respective biodegradation model is suitable. For example, a lookup table can be provided that allows the model providing unit to select based on the biodegradation habitat, for instance, based on the habitat descriptor values of the biodegradation habitat, which of the biodegradation models is suitable. However, the model providing unit 113 can also comprise or refer to an input unit to which the biodegradation model can be received, for instance, by a user selection or user input that indicates which biodegradation model should be used.

The biodegradation model is a data-driven model parameterized such that it can determine the biodegradability of the polymer based on the digital representation, in particular, based on the polymer physicochemical parameters indicative of the physicochemical characteristics associated with the polymer. In a preferred embodiment, the data-driven model refers to a machine learning model, for instance, utilizing regression model based algorithms or classifier model based algorithms. A regression model based algorithm can be based on any of a neural network algorithm, a Linear Regression algorithm, a LASSO algorithm, a Ridge Regression algorithm, a MARS algorithm, a Random Forest algorithm, and a Boosted Trees algorithm. A classifier based model algorithm can be based on any of a Random Forest algorithm, a Logistic Regression algorithm, and a SVM algorithm. The inventors have found that for most applications, in particular, Linear Regression, Random Forest, Neural Network and MARS based algorithms are suitable.

The biodegradation model can be trained, for instance, utilizing training apparatus 130. In particular, the training apparatus 130 comprises a training data providing unit 131 for providing training data for training the data-driven based biodegradation model. The training data comprises a) polymer physicochemical parameters of a plurality of training polymers, and b) biodegradabilities associated with each training polymer for one or more different habitats. Preferably, in the training data the biodegradability provided for each training polymer refer to a biodegradability that is measured in accordance with the same measurement method. Generally, the training data can be designed to cover a predetermined habitat space of a to be trained biodegradation model, wherein the habitat space is defined by the value ranges of the respective habitat descriptors for which the biodegradation model shall be trained. For example, the training data can be designed to cover predetermined polymer types for a predetermined habitat. Known methods for designing and optimizing training data for a predetermined habitat space can be utilized such that the habitat space is well covered with training data and that random outliers are avoided.

Further, the training apparatus 130 comprises a model providing unit 132 adapted to provide a data-driven based trainable biodegradation model, for instance, a biodegradation model comprising parameters that can be set during the training process for training the biodegradation model. For example, a trainable biodegradation model can already be stored on a storage unit to which the model providing unit 132 can have access for providing the same. Moreover, the training apparatus 130 comprises a training unit 133 for training the provided data-driven based biodegradation model based on the provided training data. In particular, the training can refer to varying the parameters of the biodegradation model based on the respective training data until the biodegradation model is adapted to determine a biodegradability of a polymer based on a digital representation. Generally, any known training algorithms for training data-driven, in particular, machine learning based models can be utilized. Preferably, during the training of the biodegradation model also the physicochemical parameters of the polymer that have the most influence on the biodegradability in the respective habitat are determined and the model is then trained based on these most influential physicochemical parameters. For determining these most influential physicochemical parameters, for example, cluster analysis or PCA analysis tools can be utilized. In particular, the physicochemical parameters can be utilized to determine the application space of the training data, wherein the application space is then defined by the physicochemical parameters of the polymer and the habitat descriptors that are covered by the data. The determination of the most influential physicochemical parameters and/or habitat descriptors can then be performed as a dimension reduction of the application space. Then algorithms for optimizing the training data in the application space can be applied, for instance, to cover the application space with as few training data as possible.

The training apparatus 130 then comprises a trained model providing unit 134 that is adapted to provide the trained biodegradation model, for instance, to a storage unit on which respectively trained biodegradation models for different habitat and/or different types of polymers are stored. However, the trained model providing unit 134 can also be adapted to directly provide the trained biodegradation model, for instance, to the biodegradation model providing unit 113 of apparatus 110.

In all cases, the biodegradation model providing unit 113 is then adapted to provide a suitable trained biodegradation model to the property determination unit 114. The determination unit 114 can then utilize the biodegradation model and the provided digital representation for determining the biodegradability. In particular, the determination unit 114 can be adapted to utilize the polymer physicochemical parameters indicated by the digital representation as input to the biodegradation model that has, as already described above, been trained to then provide as output a determination for the biodegradability for which it has been trained. An output unit referring, for instance, to a display, can then be adapted to output the determined biodegradability. However, the output unit can additionally or alternatively be adapted to provide the determined biodegradability to a database 140 for storing the polymer in association with the determined biodegradability for future usage. In particular, the output unit can be adapted, if for different polymers biodegradabilities have already been determined and, for instance, been stored on the storage unit, i.e. database 140, to select a respective polymer based on predetermined criteria with respect to the biodegradability. The output unit can then be adapted to provide and/or output the selected polymer and its biodegradability. This is in particular suitable in cases in which a user searches for a polymer with a specific biodegradability in one or more habitats, from a plurality of candidate polymers.

Optionally, the apparatus 110 can comprise the control unit 115 that is adapted to provide control signals based on the determined biodegradability for controlling a production process of a production system 120. In particular, it is preferred that the control unit 115 is adapted to receive a target biodegradability for a polymer and to compare the received target biodegradability with the determined biodegradability and to provide the control signal depending on the comparison, preferably, to provide control signals that indicate the usage or production of the polymer for which the biodegradability has been determined. Moreover, the control signals can be indicative of a machine executable synthesis specification of the polymer for which the biodegradability has been determined, when the result of the comparison refers to the determined biodegradability being within a predetermined range around the target biodegradability. However, the control unit 115 can also be adapted to control the production process of another product based on the determined biodegradability, for instance, to provide control signals indicative of a machine executable synthesis specification for another product utilizing or comprising the respective polymer. Moreover, the control unit 115 can provide control signals for controlling a habitat for biodegrading a polymer, for instance, in a waste management facility. For example, a target biodegradability can be met for specific habitat descriptors and the control unit 115 can then be adapted to provide control signals that control the facility such that these habitat descriptor values are met.

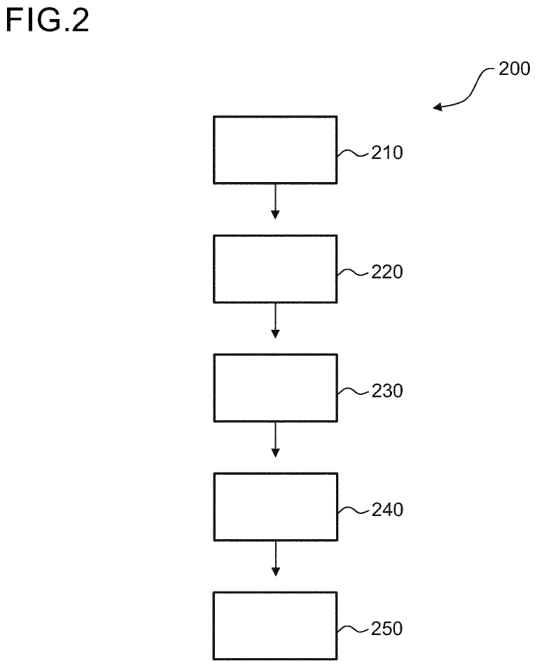

FIG. 2 shows schematically and exemplarily a flow chart of a method for determining a biodegradability of a polymer. The method 200 comprises a first step 210 of providing a digital representation of the polymer indicative of polymer physicochemical parameters of the polymer. In particular, the providing of the digital representation in this step can be in accordance with the principles described above with respect to the digital representation providing unit 111. Further, in a step 220, a biodegradation habitat indicative of habitat descriptor values of habitat descriptors influencing a biodegradation of a polymer in a respective habitat is provided. Also for this step 220, the principles described above, for instance, with respect to the habitat providing unit 112, can be applied. Further, in step 230, a biodegradation model is provided that is adapted to determine the biodegradability of the polymer based on the digital representation. As already discussed above in more detail, the providing of the biodegradation model can also refer to a selection of the biodegradation model based on the provided biodegradation habitat. Moreover, the biodegradation model is a data driven model parameterized with respect to the biodegradation habitat such that it can determine a biodegradability of a polymer based on the polymer physicochemical parameters. Generally, the steps 210, 220 and 230 can be performed in arbitrary order or even concurrently. In a following step 240, the biodegradability is determined based on the provided digital representation of the polymer and the biodegradation model. In an optional step 250, the biodegradability can then be provided, for instance, to a user interface such that the determined biodegradability for the polymer can be displayed on a display. However, in step 250, the method can additionally or alternatively comprise generating control signals that allow for a controlling of a production process of a product, for instance, the polymer or a product comprising the polymer, as already described above in more detail.

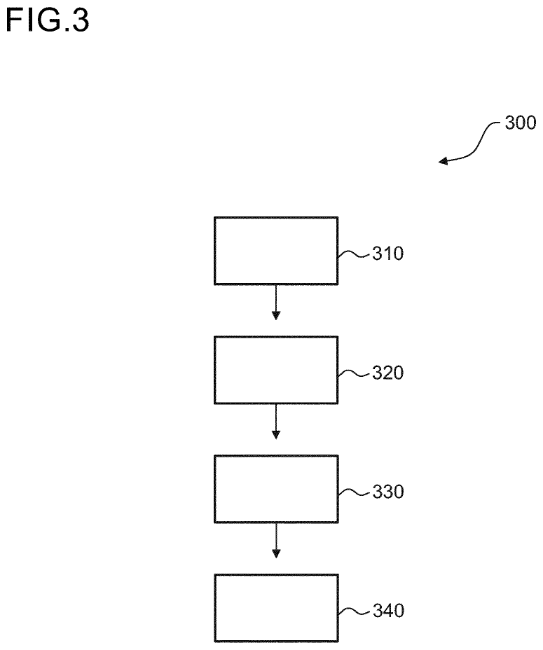

FIG. 3 shows schematically and exemplarily a flow chart of a method for training the data driven based biodegradation model utilized, for instance, in the method 200 discussed with respect to FIG. 2. Generally, the method 300 can be performed, for instance, by respective units of the training apparatus 130 as described with respect to FIG. 1. The method 300 comprises a step 310 of providing training data for training the data driven based biodegradation model. The training data comprises a) polymer physicochemical parameters of a plurality of training polymers, and b) a biodegradability associated with each training polymer in a respective biodegradation habitat, for instance, for specific habitat descriptor values. In particular, the training data can be provided in accordance with the principles described above with respect to the training data providing unit 131 described with respect to FIG. 1. The method comprises further a step 320 of providing a data driven based trainable biodegradation model, for instance, a machine learning based biodegradation model like a neural network. Generally, the step 310 and the step 320 can be performed in arbitrary order or even at the same time. The method 300 then further comprises a step 330 of training the provided data driven based biodegradation model based on the provided training data, for instance, by varying parameters in the data driven based trainable biodegradation model, such that the trained biodegradation model is adapted to determine a biodegradability of a polymer based on a digital representation of the polymer. In step 340 the trained biodegradation model can then be provided, for instance, by storing the trained biodegradation model on a storage or by directly providing the trained biodegradation model to the apparatus 130 as described with respect to FIG. 1.

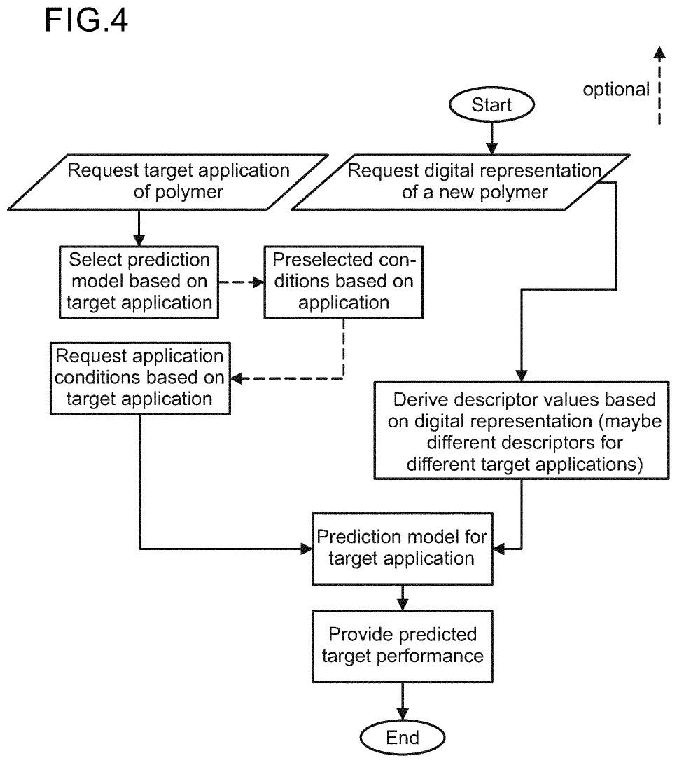

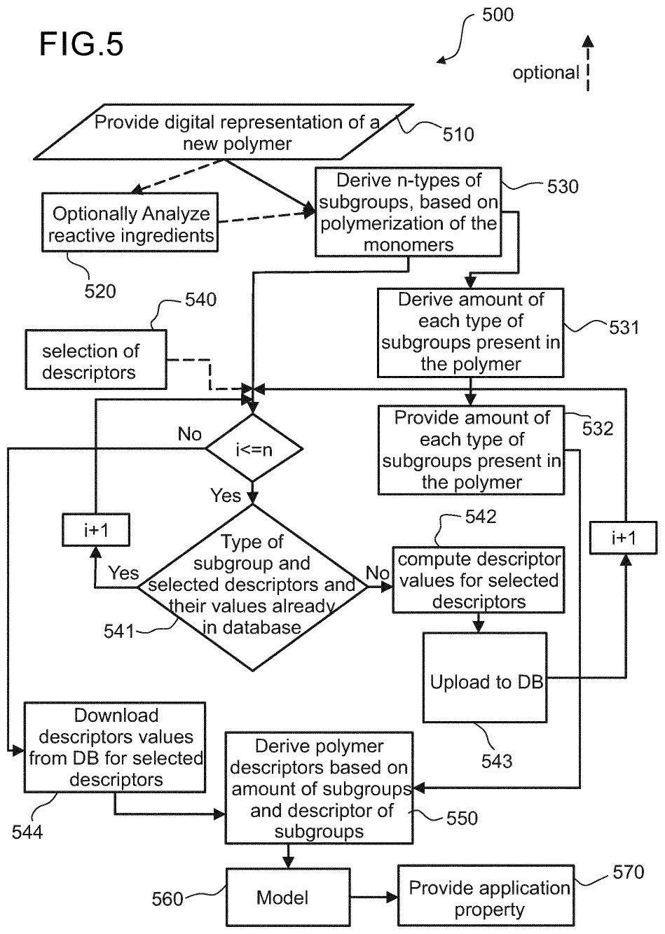

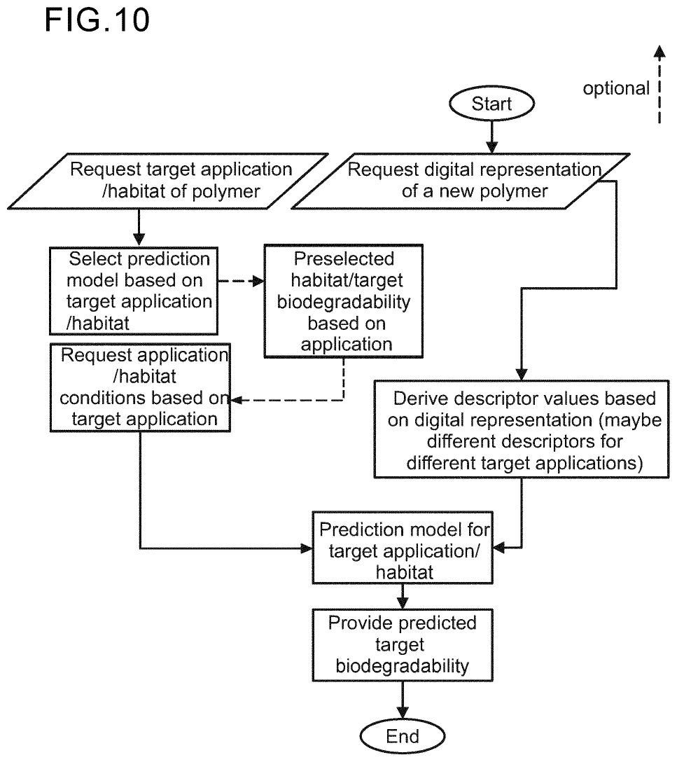

In the following, more detailed preferred examples of the above described method and the corresponding apparatus will be described. An exemplary embodiment of the method can consist of steps described in the following. A schematic and exemplary flow chart of an exemplary embodiment of the method is provided by FIG. 4. In this exemplary embodiment, the method starts with requesting, for instance, via a user interface, a digital representation of a new polymer. Further, a target application of the new polymer can be requested, for instance, also via the user interface. The target application can refer, for instance, to how the new polymer for which the digital representation is provided should be utilized in a product, or which waste treatment is expected for the polymer. The respective target application is then, for instance, indicative of a respective biodegradation habitat. For example, a list can be presented via a user interface to a user from which the user can select respective target applications and based on the respective selection further a selection of biodegradation habitats that are associated with the target application can also be provided for the user to select. Based on the target application, in particular, based on the biodegradation habitat indicated by the target application, the respective biodegradation model, i.e. biodegradation model, can be selected. Optionally, further preselected conditions indicated by the selected biodegradation model can be requested. For example, the biodegradation model can be adapted to utilize further descriptors, for instance, optional habitat descriptors, application constraints, etc. that can be requested if necessary and that might allow the biodegradation model to determine the biodegradation with a further accuracy or specifically for the constrains. Moreover, from the provided digital representation the polymer physicochemical parameter values can be derived, wherein also the polymer physicochemical parameters can depend on the provided target application. Further details on the possibilities of deriving polymer physicochemical parameter values will be described in the following with respect to FIG. 5. Utilizing the selected biodegradation model and the derived physicochemical parameter values allows then to provide a respective determined biodegradability, i.e. a respective target performance. FIG. 10 shows schematically and exemplarily the same method specific for the target technical application property being a biodegradation.

FIG. 5 shows schematically and exemplarily a preferred method 500 for deriving physicochemical parameter values, in this example referring to polymer descriptors, from a digital representation of a new polymer. In a first step 510 a digital representation of the polymer is provided. The digital representation can directly comprise the polymer descriptors, wherein in this case the steps shown in FIG. 5 until step 550 can be omitted. However, in many cases the polymer descriptors first have to be determined based on the provided digital representation referring in such a case, for example, to a recipe for the synthesis of the polymer, or to a chemical representation of the polymer indicating the chemical components and bonds in the polymer. In this step 510 the digital representation can comprise any one or more of the following information: an amount of monomer components; an amount of non-monomer components, like initiators, fillers, additives; a reaction condition, like temperature, vessel, pressure, stirring rate; condition profile, e.g. temperature profile, pH value, solvents; feed profiles; type of polymerization, e.g. radical, cationic, anionic, polycondensation, polyaddition, polyether formation; post-processing, like amount of components, conditions, as well as temperature and feed profiles; type of post-processing, e.g. radical, cationic, anionic, polycondensation, polyaddition, polyether formation; chemical information on components, like mixtures, connectivity of non-polymeric pure compounds, composition of polymeric pure compounds on the basis of subgroups, connectivity of the monomers associated to the subgroups in the polymeric pure components; for block-copolymers also information, in which block each monomer and reactive prepolymer is incorporated; for structures/layered materials and composites also information in which phase/layer each component is included. If such information is not provided by the digital representation directly, in optional step 520, the reactive components and subgroups can also be derived from the digital representation, for example, from the recipe.

If the provided information indicates the presence of a mixture, then in a following step the mixture is decomposed into its pure components and each polymer component is treated as input polymer. Moreover, the polymer composition can also be transformed into mol %, wt %, vol % or, absolute mol, if necessary.

In the next step 530 the polymerizable components can be transformed into subgroups, e.g. repeating units, and the subgroups are determined as different types. For example, polymerizable subgroups can be determined based on connectivity information of non-polymeric pure compounds by using SMARTS, for instance, via KNIME workflow. Also connectivity information of all possible subgroups can be derived from connectivity information of non-polymeric pure compounds by using reaction SMARTS, for example, also via KNIME workflow

After the subgroups and their types have been determined, in step 540 the type of descriptors that should be utilized can be provided. However, the descriptors can also be determined without first selecting the type of the subgroups. In order to decrease the computational resources for the method it is preferred that in a step 541 it is determined whether subgroup descriptors associated with a respective type of subgroup are already stored in a database, for example, if entries for subgroups with identical connectivity information already exist in the database. If this is the case the respective associated subgroup descriptor can be directly downloaded, for example, in step 544. If the determined type of subgroup is not stored on the database the subgroup descriptors that are associated with a respective type of subgroup can be determined, for example, in step 542. For example, either a 3D structure of respective types of subgroups can be derived based on connectivity information and an automatic computation of subgroup descriptors can be started using, for instance, a computer cluster, or already existing machine-learning determinations can be utilized as subgroup descriptors. Generally, it is preferred that if computations on new subgroups are necessary, in step 543, the results are stored in the database after the computations are finished. Optionally further subgroup descriptors can be provided from a topological analysis of the subgroups, a quantum chemical computation, a molecular dynamics computation, coarse-grained methods, finite-element computations and kinetic simulations. In particular, polymer reaction engineering methods can be used to derive subgroup descriptors that allow to take into account a microstructure of the polymer.

In step 531 the amount of subgroups, i.e. of each type of subgroup, is determined, for example based on the provided recipe information for the polymer and provided in step 532. For example, the amount can be determined by counting an amount of polymerizable groups per polymerizable component, optionally, including prepolymers. In this case, information on polymerizable groups can be derived from non-polymeric components and the such determined amount can be added to a count of the number of, optionally, non-polymerized, polymerizable groups of the subgroups for polymeric components based on the composition of the polymeric components to determine a resulting amount. Further, it is preferred that the amount of polymerizable groups originating from agents used for postprocessing after polymerization is removed from the resulting amount.

However, although it is preferred that the polymer descriptors are derived from polymer subgroups, in other embodiments of the invention the polymer descriptors can also be derived in other ways, for instance, by directly determining the polymer descriptors from the complete polymer. Moreover, the polymer descriptors for respective polymers can also be already stored on a storage unit such that the deriving of the polymer descriptors from a digital representation of the polymer can refer to determining from the digital representation information on the polymer that allows to access the database and retrieve the corresponding polymer descriptors.

Optionally the derived amounts of subgroups can be used for a further interpretation of the polymer composition. For example, a total number of polymerized functional groups, e.g. double bonds, amine groups, alcohols groups, thiol groups, carboxylic acid groups, isocyanate groups, epoxide groups, and formed functional groups, e.g. amid groups, ester groups, thioester groups, urea groups, urethane groups, thiourethane groups, ether groups, can be determined. Also the molar weighted total number of polymerized functional groups, the mass weighted total number of polymerized functional groups, the total number of residual functional groups, e.g. double bonds, amine groups, alcohol groups, thiol, groups, carboxylic acid groups, isocyanate groups, epoxide groups, the molar weighted total number of residual functional groups, the mass weighted total number of residual functional groups, the sum of all residual functional groups, the ratio between functional groups after polymerization, the number of crosslinks in polymer, the molar fraction of crosslinks in polymer, optionally, with mass-weighting as well, the average number of atoms per subgroup, optionally, per weight as well, the average number of non-H-atoms per subgroup, optionally, per weight as well, the average number of bonds per subgroup, optionally, per weight as well, the average number of bonds between non-H-atoms per subgroup, optionally, per weight as well, the average number of rotors per subgroup, optionally, per weight as well, the average number of rotors between non-H-atoms per subgroup, optionally, per weight as well, the average number of rings per subgroup, optionally, per weight as well, the average polar surface areas per subgroup, optionally, per weight as well, the average refractivity per subgroup, optionally, per weight as well, the total number of blocks, the molar size of first block, the molar size of last block, the HLB value of polymer, optionally, with area weighted HLB value, the HLB value of block with lowest HLB value, optionally, with area weighted HLB value, the HLB value of block with largest HLB value, optionally, with area weighted HLB value, the HLB value of first block, optionally, with area weighted HLB value, the HLB value of last block, optionally, with area weighted HLB value, the mass of first block, the mass of last block, the area of block with lowest HLB value, the area of block with largest HLB value, the difference of the HLB values of the blocks, optionally, with area weighted HLB value, the hydrophilic area of the polymer, the lipophilic area of the polymer, the number of arms for ring-opening-polymerization, or the length of arms for ring-opening-polymerization can be determined.

In step 550 the determined amount and type of the subgroups and the associated subgroup descriptors can be utilized to compute the polymer descriptors. For example, the polymer descriptors can be determined by one or more of molar weighted, e.g. arithmetic, harmonic or logarithmic, averaging, mass weighted, e.g. arithmetic, harmonic or logarithmic averaging, volume weighted, e.g. arithmetic, harmonic or logarithmic, averaging, surface area weighted, e.g. arithmetic, harmonic or logarithmic, averaging of the associated descriptors of the subgroups. Moreover, the polymer descriptors can be determined by determining from the associated subgroup descriptors one or more of a molar weighted standard deviation, a mass weighted standard deviation, a volume weighted standard deviation, a surface area weighted standard deviation, a molar weighted maximum value, a mass weighted maximum value, a volume weighted maximum value, a surface area weighted maximum value, a molar weighted minimum value, a mass weighted minimum value, a volume weighted minimum value, a surface area weighted minimum value, a molar weighted sum, a mass weighted sum, a volume weighted sum, a surface area weighted sum, and a maximal difference.

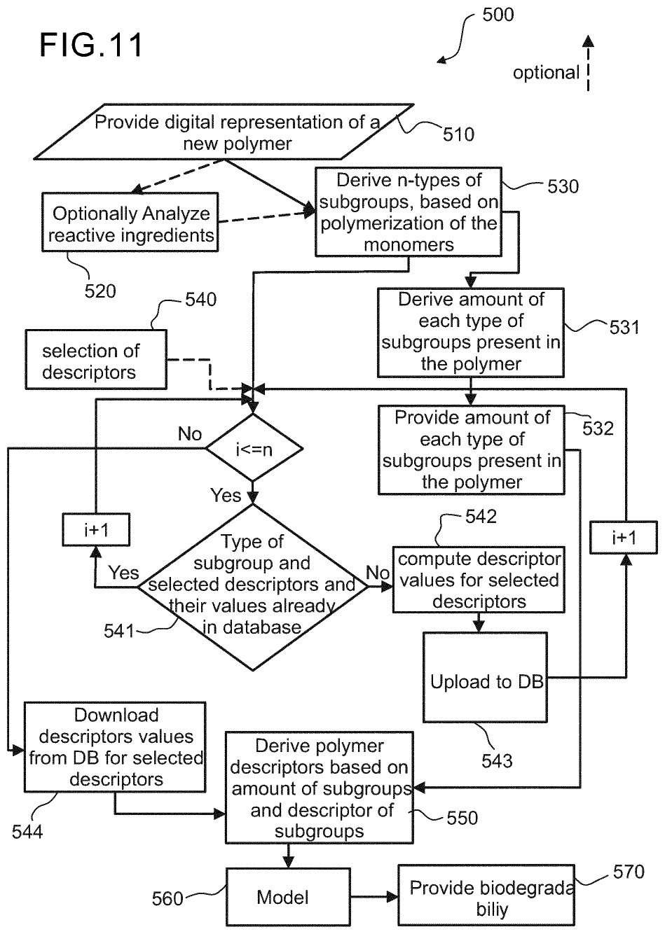

In step 560 the derived or provided polymer descriptors can then be provided to the trained biodegradation model for determining the biodegradation, for example, as described with respect to FIG. 4. The biodegradation model can be trained based on an automated statistical preprocessing of the training data, in particular, of the training polymer descriptors, for instance, utilizing feature engineering. For example, the feature engineering can comprise determining first a plurality of different polymer descriptors for the polymer, for example, based on the subgroup descriptors of the subgroups, and to preselect from this plurality of descriptors those that are with a predetermined probability relevant for the biodegradability of a polymer in a specific habitat. Based on the relevant descriptors preferably a cluster analysis is performed to identify groups of descriptors that strongly correlate. Such groups allow to select only one of the member of the groups, i.e. only one descriptor of the group, for representing the whole group of descriptors. Thus, based on the cluster analysis the number of relevant descriptors can be reduced further. The same process can optionally be performed for the habitat descriptors in order to determine habitat descriptors that are most relevant for the determination of a biodegradability of polymers in a specific habitat. Based on the remaining polymer descriptors and, optionally also based on the habitat descriptors, an application space can be determined and optimized. The application of the trained biodegradation model, for instance, to a specific habitat or polymer descriptors, can then be determined by the space spanned by the training data that forms the application space. This space can be optimized, for example, by amending the training data to cover the application space regularly, by removing strong outliers, by adding training data in parts of the space that are not yet covered, etc. This also allows to maximize the applicability space. The biodegradation model is then trained based on the optimized training data. The biodegradation model can generally refer to sparse, e.g. Splines, LASSO regression, PLS, and non-sparse, e.g. ridge regression, tree methods, kernel based methods, statistical learning models for relating the polymer descriptors to the biodegradability in a specific habitat. Moreover, the biodegradation model can further provide a reliability estimation of the determination depending on the respective used biodegradation model. In step 570 the determined biodegradability, i.e. technical application property, can then be provided to a user, for example, via a user interface. FIG. 11 shows schematically and exemplarily the same method specific for the target technical application property being a biodegradation.

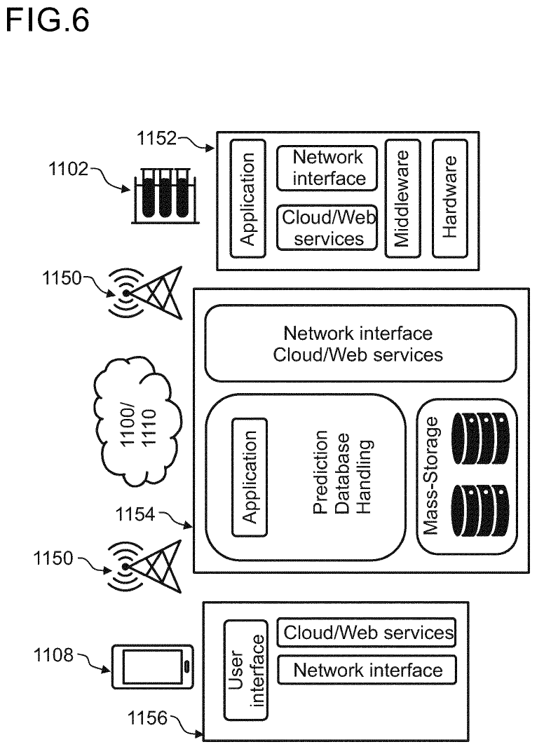

FIG. 6 illustrates a block diagram of an exemplary system architecture of an automated laboratory system 1000 for synthesizing a polymer with a laboratory equipment control device 1102, a network 1150 and the synthesis specification, i.e. recipe, module 1100/1110, and a client device 1108. The automated laboratory system includes a laboratory equipment control device layer 1152 as part of the laboratory equipment control device 1102 as well as a synthesis specification module layer 1154 associated with the synthesis specification module and a remote control or client layer 1156 associated with the client device 1108. The laboratory equipment control device layer can be split into several hierarchical layers: the hardware, the middleware and the interface layer. The hardware layer relates to hardware resources such as sensors and actuators, in particular for controlling synthesis of a polymer. The middleware relates to any of the known middleware for laboratory or plant synthesis operations. One example is LABS/QM, providing different abstractions to hardware, network and operating system such as low-level device control and message passing. The communication layer relates to communication protocols one the protocol may be REST, which may be implemented over different transport protocols (i.e. UDP, TCP, Telemetry) that allow the exchange of messages between the laboratory equipment control device and laboratory equipment devices. Such software architecture allows to control and monitor laboratory equipment without having to interact with the hardware.

The synthesis specification module layer 1154 may include: a mass storage layer, the computing layer, the interface layer. The storage layer is configured to provide mass storage for the data-driven biodegradation model for providing a recipe, i.e. synthesis specification, of a polymer based on a biodegradability, as described in detail above. In particular, the functions performed by the apparatus, as described above, can be provided as program code means stored on the mass storage. Furthermore, synthesis specifications for a plurality of polymers can be stored in the mass storage. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes based on target properties. Such functionalities can include determining based on a target biodegradability and the biodegradation model a digital representation of a target polymer, generating a synthesis specification from the digital representation of the target polymer, and providing the synthesis specification as control data to the laboratory equipment control device.

The interface layer may implement web services, network interfaces as UDP or TCP or Websocket interfaces. For communication with the laboratory equipment control device a REST API is implemented.

The client layer 1156 provides interfaces for end-users. For end-users, the client layer 1156 can run client side Web applications, which provide interfaces to the synthesis specification module layer 1154 or the laboratory equipment control device layer 1152. Users may be provided with a UI for selecting a target biodegradability and a biodegradation habitat for the target biodegradability, the target biodegradability may also comprise a range of biodegradability values. In other examples, the users may be provided with a UI for selecting more than one target biodegradability and respective values. The applications may be configured for users to monitor and control the laboratory equipment control device and the operation remotely. In other examples, the client device layer and the synthesis specification module layer may be integrated into one device. The alternatives described here are only for illustration purposes and should not be considered limiting.

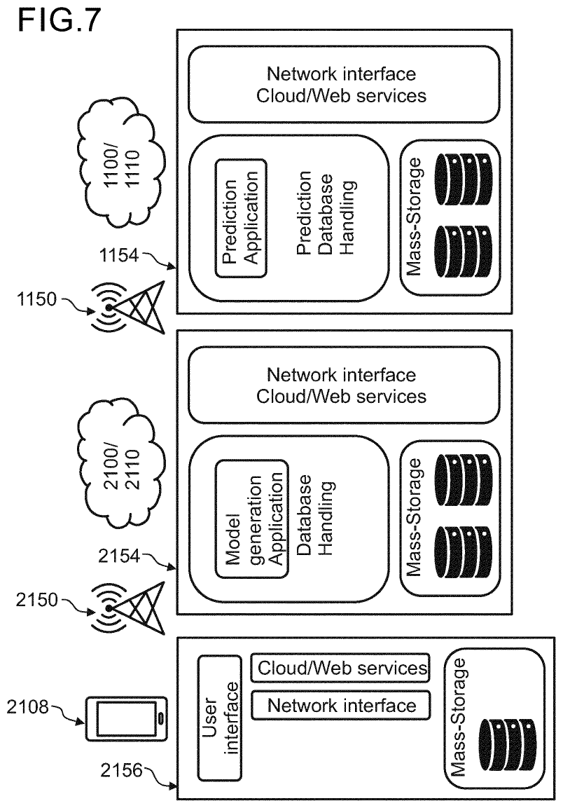

FIG. 7 illustrates a block diagram of an exemplarily system architecture of a system and apparatus for generating a biodegradation model for determining a biodegradability, a network 2150 and a model generating module 2100/2110 that can be regarded as or comprising a training model apparatus, a synthesis specification module 1100/1110, and a client device 2108. The system for generating a biodegradation model includes a model generating module layer 2154 as part of model generating module and a client layer 2156 associated with the client devices 2108.

The model generating module layer 2154 may include: a mass storage layer, a computing layer, an interface layer. The storage layer is configured to provide mass storage for the data-driven biodegradation model as described above. Furthermore, the mass storage is configured for storing synthesis specifications for polymers and measured biodegradabilities for one or more habitats. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes for generating a biodegradation model for determining a biodegradability of a polymer. Such functionalities may include receiving for at least two previously measured polymers their respective digital representations associated with a synthesis specification, measurement data of at least one biodegradability in at least one habitat for each of the at least two previously measured polymers, receiving at the model generating module the digital representation of at least one unmeasured polymer, training the model according to the above described training principles based on the digital representation of the at least two previously measured polymers, the measurement data of the biodegradability in at least one habitat for each of the at least two previously measured polymers, and, preferably, a similarity measure between the digital representation associated with the synthesis specification of each of the at least two previously measured polymers and the respective digital representation associated with a synthesis specification of the at least one unmeasured polymer, and providing via an output interface the biodegradation model for the biodegradability. The model generating module layer may be configured for deploying the generated model and the synthesis specification database to the synthesis specification module layer. This may include storing the generated model and the synthesis specification database in the mass storage devices associated with the synthesis specification module.

The model generating module layer may further be configured for determining a digital representation of the polymer associated with the synthesis specification from the synthesis specification. The digital representation may include a set of polymer physicochemical parameters and polymer physicochemical parameter values associated with a synthesis specification of each measured polymer. One way of deriving these polymer physicochemical parameters can be to apply the SMILES algorithm or any other already above described principle. In the case, where the model is generated based on the digital representation derived from the recipe, a relation between the synthesis specification and the physicochemical parameters may be stored in the mass storage devices associated with the model generating module. In such cases, deploying the model comprises providing that relation.

The interface layer may implement web services, network interfaces as UDP or TCP or Websocket interfaces. For communication with the client device a REST API is implemented in this example. The client layer 2156 provides access to mass storage devices, that contain synthesis specifications for polymers, and for at least two polymers at least one biodegradability. The client layer further provides an interface for end-users. For end-users, the client layer 2156 may run client side Web applications, which provide interfaces to the model generation module layer 2154 or the mass storage devices associated with the client layer. Users may be provided with a UI for selecting a test method and/or habitat for which the biodegradability shall be determined. The user may further be provided with a UI for selection of the synthesis specification data. The user interface may also provide an option for uploading the selected data to the model generating module layer and optionally an option to initiate model generation.

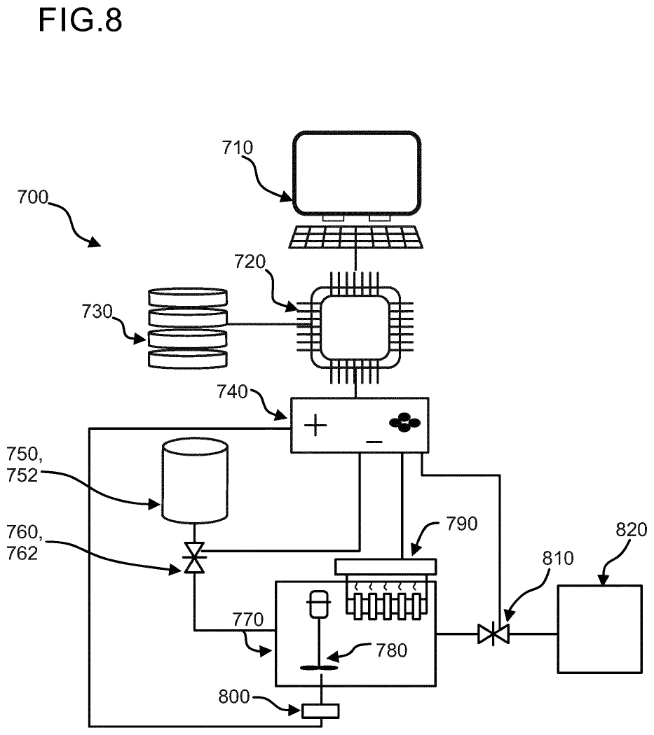

FIG. 8 shows an exemplary system 700 for producing a chemical product based on a synthesis specification generated according to the invention. In this example the system comprises a user interface 710 and a processor 720, associated with a control unit 740. The user interface 710 and the processor 720 can be associated with or realized in accordance with the principles described above, in particular, can be adapted to perform a computer implemented method to determine a target polymer and/or synthesis specification based on a determined biodegradability, as described above. The control unit 740 is, for example, configured for receiving control data generated according to the invention as described above, in particular, to receiving control data generated based on a synthesis specification of a polymer comprising a target biodegradability. In this example the control data is provided from a data base 730, in other examples, however the control data can also be provided from a server or any other computational unit for distributing data. Vessels 750, 752 each contain a component of the chemical product, for example, pre-polymers, catalysts, etc. In general more than two vessels are present, however, in this example for illustrative purposes only two are shown. Valves 760, 762 are associated with vessels 750, 752. Valves 750 and 752 can be controlled to dose appropriate amounts of each component into reactor 770, according to the synthesis specification. A motor 800 of a mixer 780 may also be controlled by the control unit according to the synthesis specification. An optional heater 790 may also be controlled according to the synthesis specification. Finally, an exit valve 810 in fluid communication with the reactor may be controlled by the control unit to provide the chemical product to a container or test system 820.

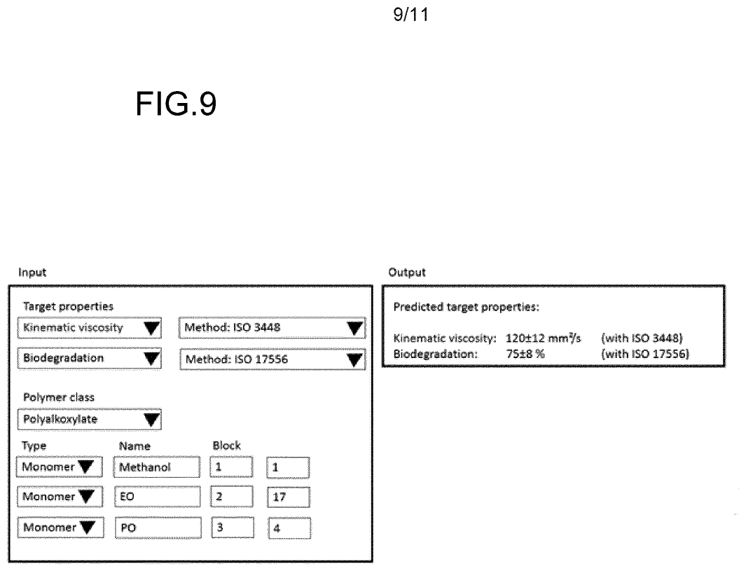

FIG. 9 shows exemplarily and schematically a possible user interface for interfacing, for example, with a processor performing the above described method for determining the biodegradability of a polymer. In this example, an input screen is shown on the right. The input screen allows for a definition of a polymer for which the biodegradability should be determined. In this case as polymer class a polyalkoxylate is defined, for example, by using a dropdown menu. Moreover, a further field is provided for further optional specifications and inputs. In this example, the polymer is then further defined below by defining components of the polymer in form of a component type and associated component, amount and block. An exemplary further definition of polyalkoxylate is provided in the FIG. 9. Additionally the input screen allows in this example to input which application properties should be determined, in this case a biodegradability and a kinematic viscosity. An additional application property can for example be determined based on a respective prediction model for the respective additional application property or by any other known method. Further, the input screen can allow to input the type of habitat. However, this can also be omitted, if the habitat can be derived from other information, for example, as in this case from the selected measurement method for the biodegradability only applicable to waste water. Generally, the input can also refer to further information, for example, to defining habitat descriptors, intended applications, measurement method, etc. For this example, it can be shown that a good prediction accuracy can be achieved when utilizing as polymer descriptor types a descriptor referring to a molar weight of the polymer, a descriptor referring to an amount of subgroups, and a descriptor referring to a hydrophiliy of the polymer. The values for such polymer descriptors can then be determined in accordance with the above described principles for the defined polymer. An exemplary output screen is shown on the right of FIG. 9. In this case the output screen provides the result of the determination of the biodegradability for the polymer and habitat defined on the input screen using the above defined polymer descriptors. For this example, the result states that for the defined polyalkoxylate 75±8% are biodegraded after 28 days in waste water and also a respectively determined kinematic viscosity is provided.

Generally, the invention refers, for instance, to a method for determining biodegradability of a new polymer. For example, in a first step a digital representation of a polymer can be provided. The digital representation may be a recipe, a structural formula, a brand name, CAS number, etc. In an optional step a target application of the new polymer may be provided. In a second step, a habitat can be selected. In this context, the term “habitat” is the biological environment in which the biodegradability shall be assessed. Dependent on the habitat, other parameters can also be relevant and then provided. In an embodiment, the habitat can be selected based on the target property. For example, for personal care products like shampoos it is generally desired that they decompose in the waste water. Consequently, for this example the automatic selection would select waste water as the habitat.

Generally, the biodegradation models can be based on habitat descriptors that are predominant for the habitat. Consequently, different biodegradation models can be chosen based on the input habitat. Thus, in a preferred workflow a biodegradation model is selected based on the habitat. Upon selection of the model the inputs of the model, the model can indicate that further inputs are necessary, for example, further polymer physicochemical parameters, etc. and the further input can be requested. This may be done by providing a list of the required parameters that should be provided. From the digital representation of the polymer physicochemical parameter values are derived, which form an input to the model. Based on the habitat descriptors and the polymer physicochemical parameters a measure for the biodegradability is determined and provided. In an optional step, the representation of the biodegradability can be selected. In this case, the output is based on the selection. Potential representations of the biodegradability may be one or more of a mineralization referring to information whether the polymer fully mineralizes or not or a time until the mineralization is achieved, a biotransformation referring to an alteration in the chemical structure resulting in the loss of a specific property of the polymer, e.g. toxicology, or time until this is achieved, a half-life referring to the time until 50% of the polymer are decomposed. Prominent habitats are Marine, waste water, and soil. For marine the following parameters can have an influence on the biodegradation: salt concentration, sediments, water temperature, bacterial cultures, etc. In some examples, the marine habitat descriptors can be stored in a database together with the geolocation. In that case the geolocation can be entered and the values of the parameters related to this geolocation can be retrieved from a database. For waste water, the following parameters can have an influence on biodegradation: Temperature, bacteria population, bacteria type, enzyme concentration, enzymes. For soil the following parameters can have an influence on biodegradation, temperature, bacteria population, bacteria type, enzyme concentration, enzymes.

In the following some examples of results of the determining of a biodegradability utilizing a biodegradation model trained and provided as described above are provided. For each example, the biodegradation model was trained for a specific class of polymers with a training data set comprising between 50 and 100 different polymers in the polymer class and respective biodegradations in a respective habitat. Moreover, each model was trained to determine a biodegradability as percentage of polymer material converted to CO₂ after 28 days based on the calculated theoretical oxygen demand of the polymer material assuming a total carbon conversion. Further, the model utilized for polyalkoxylates in waste water was trained in accordance with the standard OECD301-Test and the model utilized for polyesters in soil was trained in accordance with ISO 17556 for better comparability.

In a first example, the biodegradation model has been trained for polyalkoxylates in waste water and was based on a two-step approach in which in a first step a Random Forest model was trained and used to select polymer descriptors that are then utilized in a second step in a trained Linear Regression model for determining the biodegradability. The trained biodegradation model, in this case, the linear Regression Model, has then been applied to Plurafac LF 221 utilizing respective physicochemical characteristics. The output of the biodegradation model resulted in a biodegradation of 91%, meaning that 91% of the polymer material has been converted to CO₂ based on the calculated theoretical oxygen demand of the polymer material assuming a total carbon conversion, wherein a measured biodegradation for this polymer is reported as lying above 60%.

In a second example, the biodegradation model has been trained for polyalkoxylates in waste water in the same way as in the first example. In particular, for both exampled the same biodegradation model can be used. The biodegradation model has then been applied to Pluriol E 200 utilizing respective physicochemical characteristics. The output of the biodegradation model resulted in a biodegradation of 96%, meaning that 96% of the polymer material has been converted to CO2 based on the calculated theoretical oxygen demand of the polymer material assuming a total carbon conversion, wherein a measured biodegradation for this polymer is reported as lying above 70%.

In a third example, the biodegradation model has been trained for amine terminated polyalkoxylates in waste water in the same way as in the first example. In particular, for both exampled the same biodegradation model can be used. The biodegradation model has then been applied to Jeffamine D 230 utilizing respective physicochemical characteristics.

The output of the biodegradation model resulted in a biodegradation of 7%, meaning that 7% of the polymer material has been converted to CO2 based on the calculated theoretical oxygen demand of the polymer material assuming a total carbon conversion, wherein a measured biodegradation for this polymer is reported at 7.2%.

In a fourth example, the biodegradation model has been trained for polyester in soil. In this example, the trained biodegradation model is based on a trained Partial Least Squares model. The biodegradation model has then been applied to Lupraphen 1619/1 utilizing respective physicochemical characteristics. The output of the biodegradation model resulted in a biodegradation of 49%, meaning that 49% of the polymer material has been converted to CO2 based on the calculated theoretical oxygen demand of the polymer material assuming a total carbon conversion, wherein a measured biodegradation for this polymer is reported as lying above 60%.

With an expected measurement error for the measured values of +/−10% for waste water and +/−10% for soil, the results of the trained biodegradation models lie within a suitable accuracy range for the intended application. Thus, a training using a training data set with only 50 polymer data points already provides a suitable accuracy. Utilizing a data set with more polymers might even lead to a higher accuracy. A biodegradation model can thus be trained accordingly depending on an accuracy suitable for a respective application.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of the polymer physicochemical parameters and the biodegradation model, the determining of the biodegradability, the providing of the biodegradability, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit.

Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, main-frame computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refer to a method for determining a biodegradability for a polymer. A digital representation of the polymer indicative of physicochemical characteristics of the polymer is provided. Further, a habitat is provided that is indicative of habitat descriptor values influencing a biodegradation of a polymer. The habitat descriptors are indicative of environmental characteristics of the habitat. A biodegradation model is provided based on the habitat, wherein the biodegradation model is adapted to determine a biodegradability of a polymer in the respective habitat, wherein the biodegradation model is a data driven model parametrized with respect to the habitat such that it can determine a biodegradability of a polymer based on the physicochemical characteristics. The biodegradability of the polymer is then determined based on the provided biodegradation model and the digital representation of the polymer.

1. A computer implemented method for determining a biodegradability usable for validating a biodegradation for a polymer, wherein the method comprises:
providing a digital representation of the polymer indicative of or associated with physicochemical characteristics of the polymer,
providing a biodegradation habitat, wherein a biodegradation habitat is indicative of habitat descriptor values of habitat descriptors influencing a biodegradation of a polymer in the respective habitat, wherein the habitat descriptors are indicative of environmental characteristics of the habitat,
providing a biodegradation model based on the provided biodegradation habitat, wherein the biodegradation model is adapted to determine a biodegradability of a polymer in the respective biodegradation habitat, wherein the biodegradation model is a data driven model parametrized with respect to the biodegradation habitat such that it can determine a biodegradability of a polymer based on the physicochemical characteristics, and
determining the biodegradability of the polymer based on the provided biodegradation model and the digital representation of the polymer.

2. The method according to claim 1, wherein the method further comprises providing a biodegradation test method, wherein the provided biodegradation test method is indicative of a standardised biodegradation test method for determining experimentally a biodegradation of a chemical, wherein the biodegradation model is further provided based on the provided biodegradation test method.

3. The method according to claim 1, wherein the biodegradation habitat refers to any one of a marine habitat, a waste water habitat, a limnic habitat, a compost habitat, an anaerobic habitat or a soil habitat.

4. The method according to claim 3, wherein the biodegradation habitat refers to a marine habitat and wherein the habitat descriptors refer to at least one of a salt concentration, a sedimentation type, oxygen level, location, sample depth, a water temperature, a nutrient concentration, a pH value, an environmental type and a microbial community.

5. The method according to claim 3, wherein the biodegradation habitat refers to waste water and the habitat descriptors refer to at least one of a water temperature, a microbial community, a sludge concentration, a nutrient concentration, a pH value, a test duration and an enzyme environment.

6. The method according to claim 3, wherein the biodegradation habitat refers to soil and the habitat descriptors refer to at least one of a temperature, a sand content, a pH value, a moisture content, a nutrient concentration, a microbial community and an enzyme environment.

7. The method according to claim 3, wherein the biodegradation habitat refers to compost and the habitat descriptors refer to at least one of a temperature, compost activity, a pH value, a moisture content, humidity, compost maturity, compost composition, compost origin, a nutrient concentration, a microbial community and an enzyme environment.

8. The method according to claim 1, wherein habitat descriptor values for the habitat descriptors are stored associated with respective geolocations, wherein the providing of a biodegradation habitat refers to providing a geolocation of the habitat and retrieving the habitat descriptor values for the geolocation from storage.

9. An interface method for providing an interface, wherein the interface method comprises:
receiving as input a digital representation and a habitat via a user interface and providing the received digital representation and the habitat to a processor performing the method according to claim 1, and
providing the determined biodegradability of the polymer to a user via a user interface as result, wherein the result is received from the processor performing the method according to claim 1.

10. A computer implemented training method for training a data driven based biodegradation model for parameterizing the biodegradation model, wherein the training method comprises:
providing training data associated with a predetermined biodegradation habitat, wherein the training data comprises a) digital representations of a plurality of training polymers indicative of physicochemical characteristics for each of the training polymers, and b) a biodegradability for the respective biodegradation habitat associated with each training polymer,
providing a data driven based trainable biodegradation model,
training the provided data driven based biodegradation model based on the provided training data such that the trained biodegradation model is adapted to determine a biodegradation of a polymer based on physicochemical characteristics of the polymer, and
providing the trained biodegradation model.

11. An apparatus for determining a biodegradability usable for validating a biodegradation for a predetermined polymer, wherein the apparatus comprises:
a digital representation providing unit for providing a digital representation of the polymer indicative or associated with physicochemical characteristics of the polymer,
a habitat providing unit for providing a biodegradation habitat, wherein a biodegradation habitat is indicative of habitat descriptor values of habitat descriptors influencing a biodegradation of a polymer in the respective habitat, wherein the habitat descriptors are indicative of environmental characteristics of the habitat,
a model providing unit for providing a biodegradation model based on the provided biodegradation habitat, wherein the biodegradation model is adapted to determine a biodegradability of a polymer in the respective biodegradation habitat, wherein the biodegradation model is a data driven model parametrized with respect to the biodegradation habitat such that it determines a biodegradability of a polymer based on the physicochemical characteristics, and
a determining unit for determining the biodegradability of the polymer based on the selected biodegradation model and the digital representation of the polymer.

12. An interface apparatus for providing an interface, wherein the interface apparatus comprises:
an input interface unit for receiving as input a digital representation and a habitat via a user interface and for providing the received digital representation and the habitat to the apparatus according to claim 11, and
a result interface for providing the determined biodegradability of the polymer to a user via a user interface as result, wherein the result is received from the apparatus according to claim 11.

13. A training apparatus for training a data driven based biodegradation model for parameterizing the biodegradation model, wherein the training apparatus comprises:
a training data providing unit for providing training data associated with a predetermined biodegradation habitat, wherein the training data comprises a) digital representations of a plurality of training polymers indicative of physicochemical characteristics for each of the training polymers, and b) a biodegradability for the respective biodegradation habitat associated with each training polymer,
a trainable model providing unit for providing a data driven based trainable biodegradation model,
a training unit for training the provided data driven based biodegradation model based on the provided training data such that the trained biodegradation model is adapted to determine a biodegradation of a polymer based on physicochemical characteristics of the polymer, and
a trained model providing unit for providing the trained biodegradation model.

14. A computer program product for determining a biodegradability for a predetermined polymer, wherein the computer program product comprises program code means for causing the apparatus of claim 11 to execute the method according to claim 1.

15. A computer program product for training a biodegradation model, wherein the computer program product comprises program code means for causing the apparatus of claim 13 to execute the method according to claim 10.
